(19) United States
(12) Patent Application Publication
(10) Pub. No.: US 2021/0217516 A1
(43) Pub. Date: Jul. 15, 2021

(54) SYSTEM AND METHOD OF TREATING A PATIENT BY A HEALTHCARE PROVIDER USING A PLURALITY OF N-OF-1 MICRO-TREATMENTS

(71) Applicant: INDIVIDUALLYTICS INC., West Bloomfield, MI (US)

(72) Inventors: Dennis Nash, West Bloomfield, MI (US); Steve Schwartz, Plymouth, MI (US)

(73) Assignee: INDIVIDUALLYTICS INC., West Bloomfield, MI (US)

(21) Appl. No.: 17/250,824

(22) PCT Filed: Sep. 5, 2019
(86) PCT No.: PCT/US2019/049739

§ 371 Date: Mar. 5, 2021

Related U.S. Application Data
(60) Provisional application No. 62/727,296, filed on Sep. 5, 2018.

(51) Int. Cl. G16H 40/20; G16H 10/60; G16H 50/70; G16H 15/00; G16H 40/67; G06F 9/451; G06F 16/245

(52) U.S. Cl. CPC G16H 40/20; G16H 10/60; G16H 50/70; G16H 50/20; G16H 40/67; G06F 9/453; G06F 16/245; G16H 15/00

(57) ABSTRACT

A patient treatment system includes a method that is used to actively monitor and treat a patient based on response data received from the patient as a result of a plurality of micro-treatments, and the system performs an N-of-1 statistical analysis of the response data. The data is automatically collected and obtained from the patient by virtue of the patient wearing a wearable device. The system generates a graphical user interface that includes an effectiveness display of a response level to each micro-treatment, a trendline representing a trend of the data for each micro-treatment; data scores for each micro-treatment, a confidence display of a statistical confidence associated with each data score; graphical elements representing the statistical confidence associated with each data score.

### CROSS REFERENCE TO RELATED APPLICATIONS

This application claims the benefit of U.S. Provisional Patent Application Ser. No. 62/727,296, filed Sep. 5, 2018, the entirety of which is hereby incorporated by reference.

### TECHNICAL FIELD

The present disclosure pertains to a system and method of treatment of a patient by a healthcare provider by using a plurality of N-of-1 micro-treatments.

### BACKGROUND

After many centuries and millennia of “snake oil” sales people and witch doctors offering treatments to diseases, the advent of scientist, medical professionals, and statisticians developed expensive random control trials gold standard to bring scientific rigor to validate treatment effect. When a drug or treatment works for nearly everyone, such as cures for strep throat or many pain medications, there is a high confidence that most people can be successfully treated with these treatments, i.e., population-based science.

This population-based science led to the growth of the pharmaceutical industry and many blockbuster drug successes and other medical/surgical treatments. The otherwise expensive cost of random control trials is amortized across a large number of patients, which has made these high confidence, complex studies affordable. This approach works well when the assumption is made that all humans are largely the same and will respond to treatment similarly. However, at the same time, science has learned that humans are also very different from one another, where each human has a unique genetic makeup, has a unique brain, exists in a unique environment, with different learning histories, habits, values and lifestyle, etc.

Society's more challenging diseases, such as diabetes, COPD, mental health, Alzheimer's Disease, etc., are complex and chronic. Many of these chronic diseases have beneficial treatment population effect sizes that are less than 50%, as compared to placebo or current standard of care control groups. For example, many depression medicines, on average, work for about 20% of patients, as compared to placebo, while experiencing only minimal side effects. As another example, there are currently only four FDA approved compounds for the treatment of Alzheimer's Disease. Only 4% of Alzheimer's Disease patients receive moderate or significant benefit when treated with these four compounds, as compared to placebo, while experiencing only minimal side effects.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method of using a patient treatment system to actively monitor and treat a patient. The method includes: receiving, by a computing device, first and second order response data corresponding to a respective first and second micro-treatment prescribed to a patient, where the first and second order response data represents results of the respective first and second micro-treatment for the patient at each of a plurality of intervals in time. The method also includes where the second micro-treatment occurs after the first micro-treatment. The method also includes recording the first and second order response data into a database that includes time series response data for each of the first and second micro-treatments; calculating, by the computing device: a first data score and a second data score by applying an N-of-1 statistical analysis respectively to each of the first and second order response data, where the first and second data scores statistically represent an effectiveness of the respective first and second micro-treatment; a trend of the first and second data scores; and a statistical confidence associated with each of the first and second data scores. The method also includes recording the first and second data scores into the database and generating, by the computing device, a graphical user interface on a display screen of a user device.

The graphical user interface includes at least one of an effectiveness display that displays at least one of the response level to each of the first and second micro-treatments and a trend line representing the trend of the first and second data scores; the first and second data scores and a confidence display that displays the statistical confidence associated with each of the first and second data scores; first and second graphical elements, where the first and second graphical element represent the statistical confidence associated with each of the first and second data scores. The method also includes generating, by the computing device, a graphical user interface on the display screen of the user device including at least one third micro-treatment option to be prescribed to the patient.

Another general aspect includes a method of treating a patient with a patient treatment system, the method including: receiving, by a computing device, first and X^{th} order response data corresponding a respective first and X^{th} micro-treatment prescribed to a patient, where the first and X^{th} order response data corresponds to the results of the respective first and X^{th} micro-treatment for the patient at each of a plurality of intervals in time; where the X^{th} micro-treatment occurs after the first micro-treatment; recording the first and X^{th} order response data into a database that includes time series response data for each of the first and X^{th} micro-treatments; calculating, by the computing device, a first data score and an X^{th} data score by applying an N-of-1 statistical analysis respectively to each of the first and X^{th} order response data, where the first and X^{th} data scores statistically represent an effectiveness of the respective first and X^{th} micro-treatment; calculating, by the computing device, a first-to-Nth delta representing a difference between the X^{th} data score and the first data score, where the first-to-X^{th} delta represents an amount of change of the micro-treatment effectiveness from the first to the X^{th} micro-treatment; and generating, by the computing device, a graphical user interface on a display screen of a user device, where the graphical user interface includes: a change display that displays an X-Y plot of the first data score and the X^{th} data score to graphically represent an amount of change of the micro-treatment effectiveness from the first micro-treatment to the X^{th} micro-treatment; and displaying the generated graphical user interface. Other embodiments of this aspect include corresponding computer systems, apparatus, and computer programs recorded on one or more computer storage devices, each configured to perform the actions of the methods.

Yet another general aspect includes a method of treating a patient with a patient treatment system, the method including: recording at least one health attribute and at least one health condition of a patient into a database, such that the at least one health attribute and the at least one health condition is associated with a patient profile of the patient; recording first and second order response data into a database that includes time series response data for each of a first and second micro-treatment, such that the first and second order response data is associated with the patient profile of the patient; calculating, by the computing device, a first data score and a second data score by respectively applying an N-of-1 statistical analysis to each of the first and second order response data, where the first and second data scores statistically represent an effectiveness of the respective first and second micro-treatment; recording the first and second data scores into the database, such that the first and second data scores are associated with the patient profile of the patient; calculating, by the computing device, a first-to-second delta representing a difference between the second data score and the first data score, where the first-to-second delta represents an amount of change of the micro-treatment effectiveness from the first to the second micro-treatment; recording the first-to-second delta into the database, such that the first-to-second delta is associated with the patient profile of the patient; where the database further includes another patient profile corresponding to one other patient, where the patient profile of the one other patient includes a health attribute, a health condition, first and second order response data corresponding to a first and second micro-treatment prescribed to the other patient, where the first and second order response data corresponds to the results of the respective first and second micro-treatments at each of a plurality of time intervals, and first and second data scores that statistically represent an effectiveness of each of the first and second micro-treatments for the other patient; generating, by the computing device, a graphical user interface on a display screen of a user device, where the graphical user interface includes: a change display that displays an X-Y plot of for the patient representing the first order and second order response data at each of the plurality of intervals during the respective first and second micro-treatment and that displays an X-Y plot for the other patient representing the first order and second order response data at each of the plurality of intervals during the respective first and second micro-treatment; and displaying the generated graphical user interface. Other embodiments of this aspect include corresponding computer systems, apparatus, and computer programs recorded on one or more computer storage devices, each configured to perform the actions of the methods.

In one aspect of the disclosure, a treatment system is provided for blending known population-based treatment effects (group averages) with N-of-1 measures (of the individual patient).

In another aspect of the disclosure, a treatment system is provided for blending known population-based treatment effects with N-of-1 science for displaying intervention insights and group clusters.

In yet another aspect of the disclosure, a treatment system is provided for drug and trial treatment enhancement with environmental sensor data.

Another aspect of the disclosure provides a treatment system for crowdsourcing (i.e. a model by which individuals data and/or activity) is organized to optimize the value or goods and/or services. These services include ideas and finances, from a large, relatively open and often rapidly-evolving group of individuals and their inputs) new treatment insights.

Evidence-based medicine (EBM) is the application of scientific evidence to clinical practice. In most medical trials and treatments, global evidence (“average effects” or “population-based treatment effects” measured as population means) is applied to individual patients, regardless of whether those individual patients depart from the population average. In getting drugs approved for treatment of a medical condition during clinical trials, the benefit or harm can be misleading and fail to reveal the potentially complex mixture of substantial benefits for some, little benefit for many, and harm for a few.

With nearly a 100% standard of care, a doctor's treatment of a patient having a complex chronic disease is based solely on population-based science and based on the probability of helping the most people the most based on known effects, even when known current recommended treatment only has a 1:25 population effect size. Further, the current standard of care is typically a medical assessment that occurs at a single point in time, and then a single one to twelve-month follow-up assessment in nearly all chronic health cases. Typically, this level of follow up leads to infrequent subsequent visits and assessments of treatment response. This long standing, long-interval approach reduces the opportunity to find the best or optimized treatment for each patient. Statistically, this long-interval approach creates a high number of false positives or false negative effects for chronic health care. In many cases, placebo or other non-medical treatments, e.g., exercise or diet change, would have a higher positive effect with less side effects. For many ailments, this long-interval approach not only reduces positive outcomes for individual patients, but in many cases, this reduces positive outcomes for much of the disease population. There is a big opportunity by providing more evidenced-based personalized care in more scalable, cost-effective approach for collecting data more frequently and displaying easy to understand standardized N-of-1 decision support data fast enough and often enough.

Some patients will experience more or less benefit from treatment than the averages reported from clinical trials; such variation in therapeutic outcome is termed heterogeneity of treatment effects (HTE). Identifying HTE 15 necessary to individualize treatment, since HTE reflects patient diversity in risk of disease, responsiveness to treatment, vulnerability to adverse effects, and utility for different outcomes. By recognizing these factors, customized treatments can be prescribed and documented at the individual (N-of-1) patient level to effectively determine which treatment is most effective for an individual.

These individual differences need the application of individual science, or N-of-1 statistics based off of N-of-1 trials, to have rigor or confidence. Just like population-based science, the goal with N-of-1 trials is to gain confidence in the likelihood of a true cause and effect relationship, or reduce Type 1 or Type 2 errors (false positive or false negative observations), while providing individualized treatment. In population studies, a high confidence is achieved by increasing the number of participants (a high N). For individuals (N-of-1), a study needs more measurements per treatment time period (or “segment”).

There is a need to better understand the true treatment effect on an individual (N-of-1), with a high confidence. N-of-1 (single subject) trials consider an individual patient as the sole unit of observation in a study investigating the efficacy or side-effects of different treatments. The ultimate goal of an N-of-1 trial is to determine the optimal or best intervention for an individual patient using objective data-driven criteria. However, due to the high costs associated with individualized attention to a patient, N-of-1 trials have been used sparingly in medical and general clinical settings.

Also, wide adoption has been limited due to the burden in overseeing longitudinal data collection (i.e., track the same sample at different points in time), low patient data completeness, the inability to do analysis of the data fast enough to generate impact, a lack of standards, and a difficulty in getting payment from insurance providers for this higher cost approach. These, and other challenges, continue to limit the use of this more accurate personalized scientific treatment approach. Therefore, there exists a need for a simple, fast, practical, cost effective, standardized, and reliable indicator of individual patient treatment effectiveness, or lack of effectiveness, with less decision errors (i.e., more confidence).

There is a need for diagnosing root cause issues and accurate treatment effect decision making for other complex systems, not just patients, for example, but not limited to, humans, animals, plants, smart systems, mechanical systems, computer systems, and the like.

The above noted and other features and advantages of the present disclosure are readily apparent from the following detailed description when taken in connection with the accompanying drawings.

### BRIEF DESCRIPTION OF THE DRAWINGS

FIG. 1 provides a schematic illustration of an exemplary treatment system for a treatment system for treating a patient by a healthcare provider.

FIG. 2 is a flow chart describing an example method the treatment system of FIG. 1.

FIG. 3A is a schematic illustrative graphical user interface of an exemplary chart representing a quality score of three different micro-treatments, across three segments.

FIG. 3B is a schematic illustrative graphical user interface of another exemplary chart or digital dashboard representing multiple patients, and their names, associated current micro-treatment, micro-treatment trends, recommended micro-treatments, compliance, an outcome variable being measured throughout the micro-treatments, compliance percentage, an IAQ score, and a delectable details link to allow a healthcare provide to open a.

FIG. 3C is a schematic illustrative graphical user interface of yet another exemplary chart representing a quality score of three different micro-treatments, across three segments for the patient “Raymond” represented in FIG. 3B.

FIG. 4 is a schematic block diagram illustrating patient data.

FIGS. 5-10 represent a schematic series of X-Y graphical journey maps showing depression versus quality of life for a patient over an interval of time.

FIGS. 11-15 represent a schematic series of X-Y graphical journey maps showing depression versus quality of life for four different patients over an interval of time for each of three different micro-treatment phases, i.e., Phase A, Phase, B, and Phase C.

FIGS. 16-21 represent a schematic series of X-Y graphical journey maps showing depression versus quality of life for four different treatment clusters, with each treatment cluster including 1000 patients, over an interval of time for each of three different micro-treatment phases, i.e., Phase A, Phase B, and Phase C.

FIG. 22-28 represent a schematic series of X-Y graphical journey maps showing depression versus quality of life for five different treatment clusters, with each treatment cluster including 1000 patients, over an interval of time for each cluster over three different micro-treatment phases, i.e., Phase A, Phase B, and Phase C.

FIG. 29-35 represent a schematic series of X-Y graphical journey maps showing depression versus quality of life for a single treatment cluster, as compared with a single patient from the treatment cluster, over an interval of time over three different micro-treatments, i.e., Phase A, Phase B, and Phase C.

FIG. 36-40 represent a schematic series of X-Y graphical journey maps showing depression versus quality of life for a patient over an interval of time and over three different micro-treatment phases, i.e., Phase A, Phase B, and Phase C, while showing a confidence score for the patient at each of the intervals in time, over each of the phases.

### DESCRIPTION

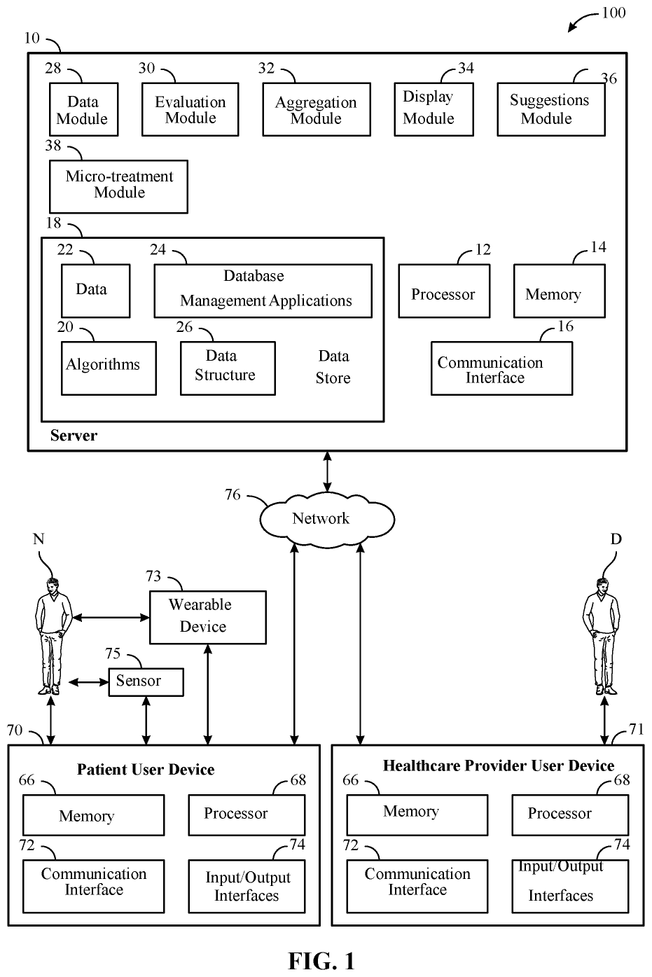

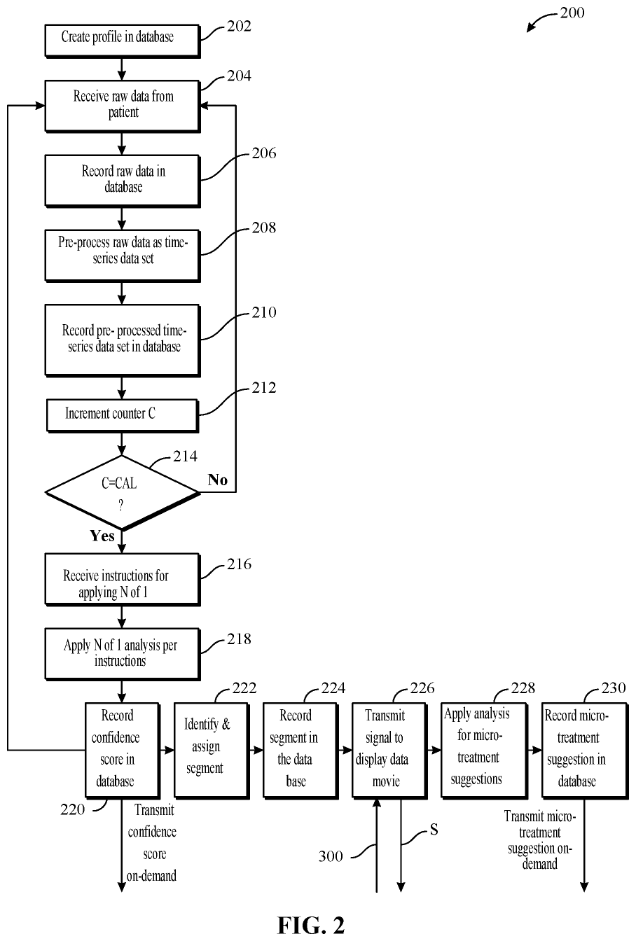

FIG. 1 shows an exemplary schematic illustration of a treatment system 100 for executing an exemplary treatment process 200 illustrated by the block diagram shown in FIG. 2. The treatment system 100 is configured to quickly and efficiently blend known population-based treatment effects (“group average science” or GAS) with individual science (N-of-1) to support individual and population health outcomes and enable better personalized care, while reducing medical system costs in the treatment of, or development of cures for, diseases, disorders, injuries, complex system problems, and the like (“ailments”). The ailments capable of being targeted by the treatment system 100 include, but should not be limited to, allergic disease, autoimmune disease, cardiac disease, dermatologic disease, endocrine disease, gastrointestinal disease, genetic disease, hematologic disease, immunodeficiency disease, infectious disease, neurologic disease, oncologic disease, pulmonary disease, renal disease, emotional issues, behavioral risk and rheumatologic disease. The disorders capable of receiving effective treatment by the treatment system 100 may include mental health disorders, such as depression, along with other complex chronic diseases, such as Alzheimer's Disease, dementia, rheumatoid arthritis, diabetes, multiple sclerosis, lupus, cancer, and the like can be realized.

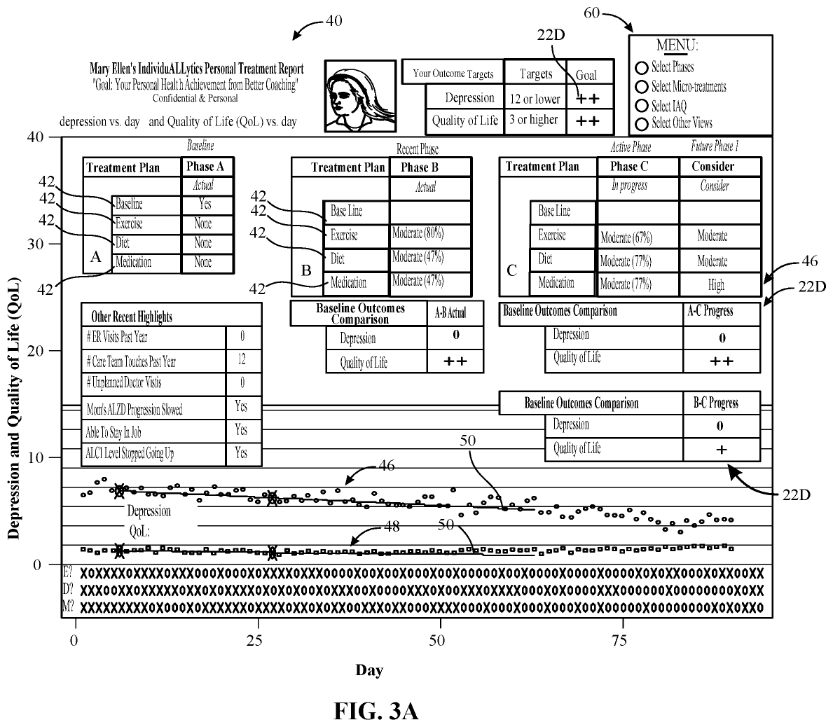

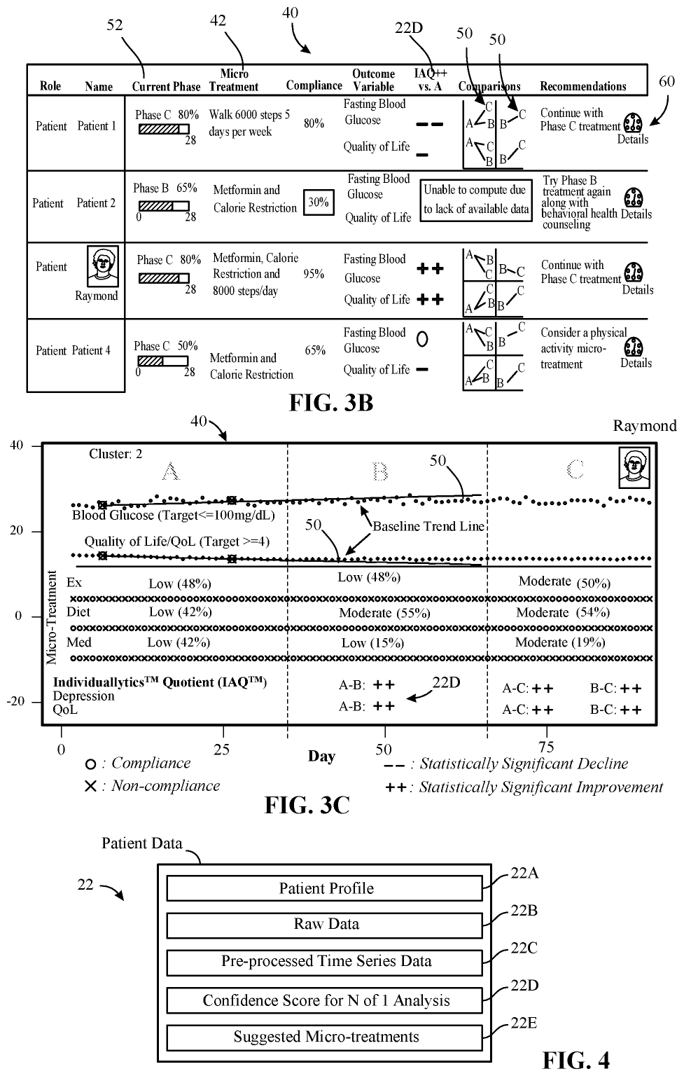

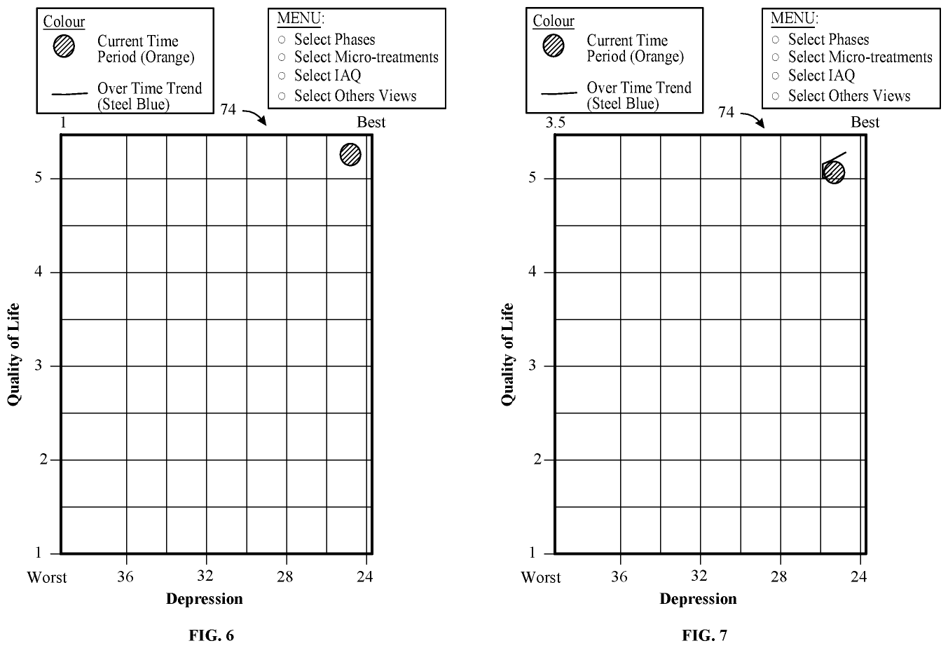

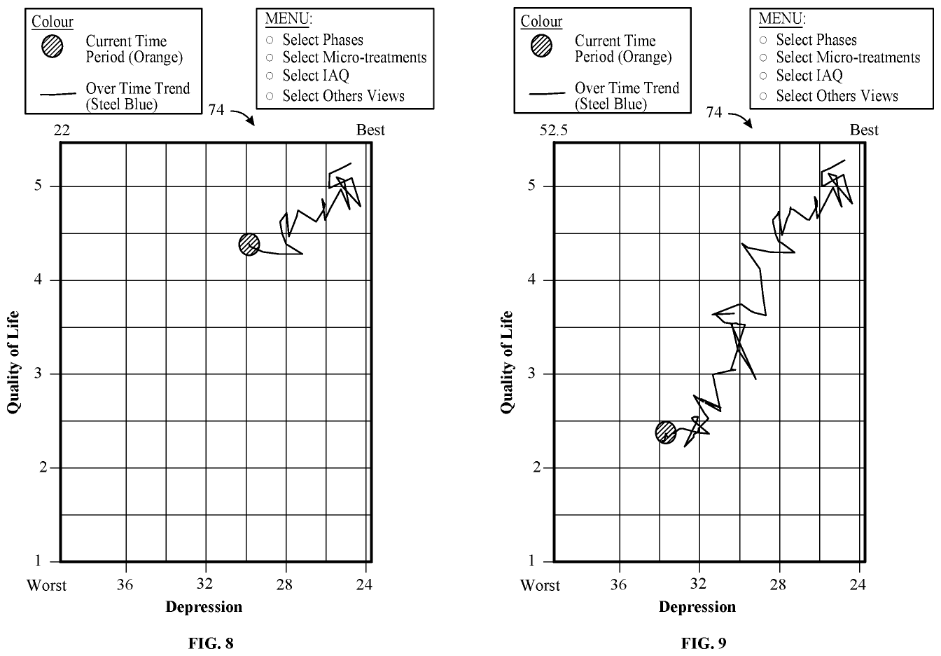

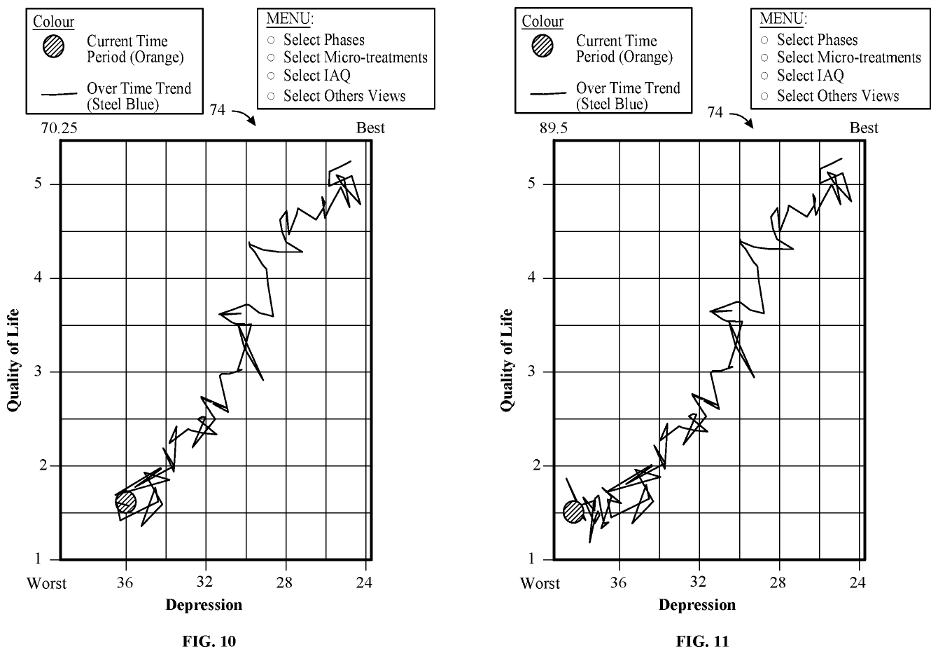

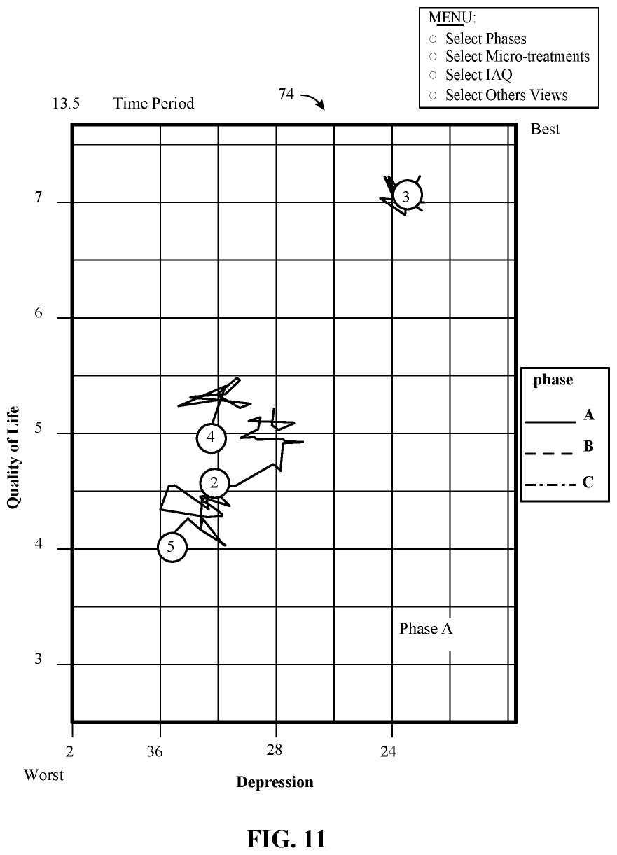

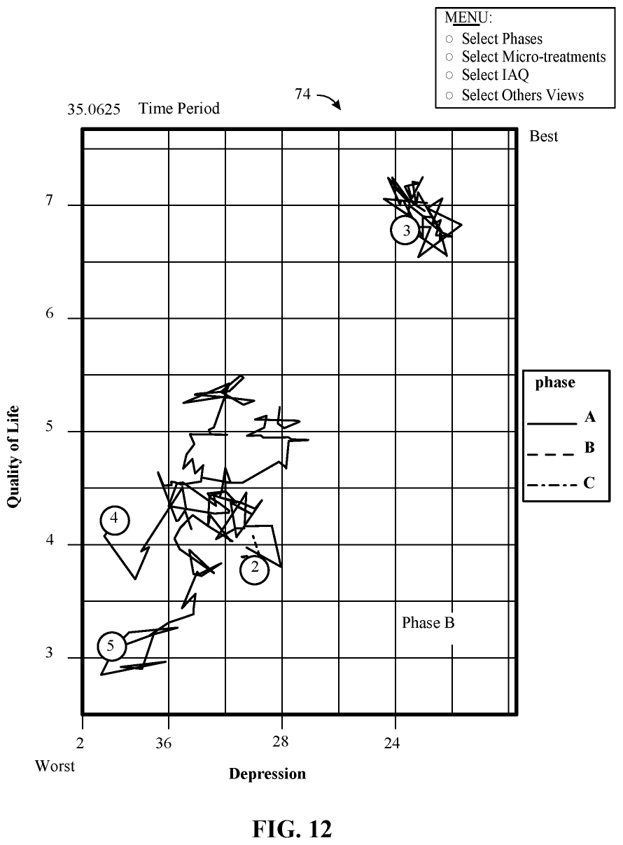

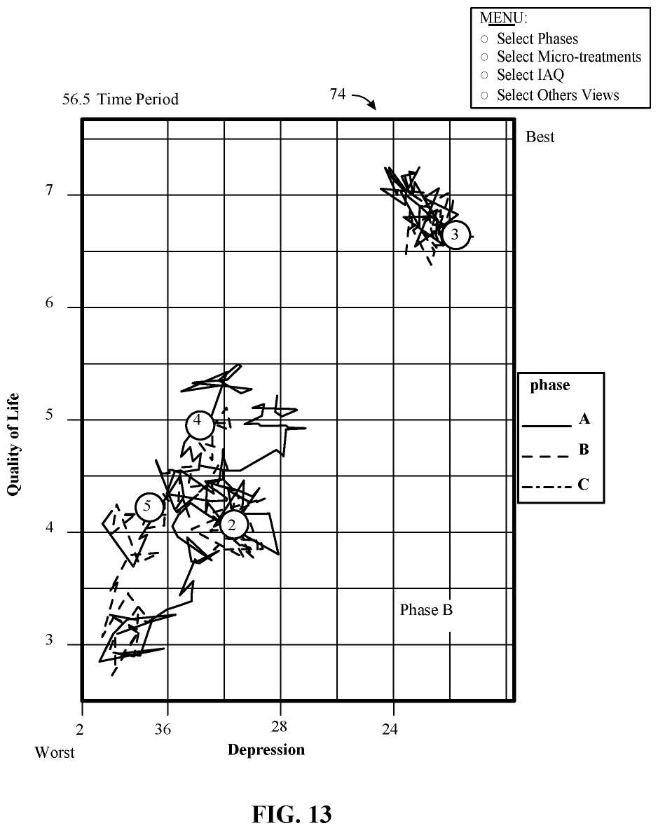

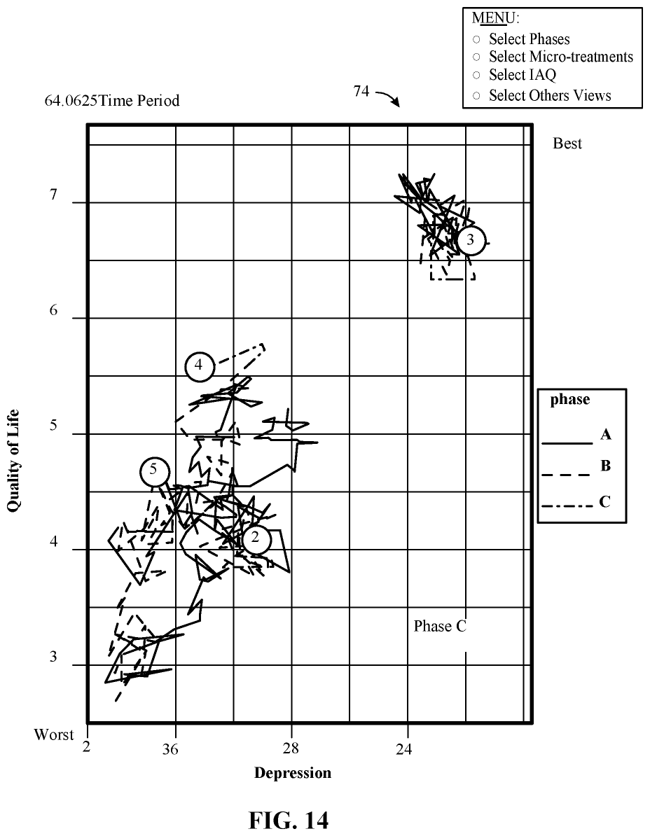

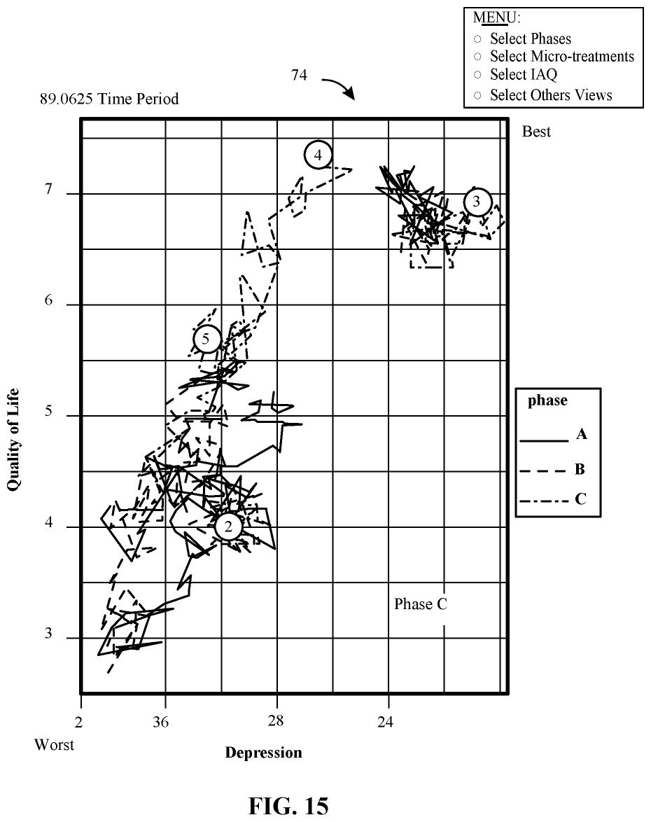

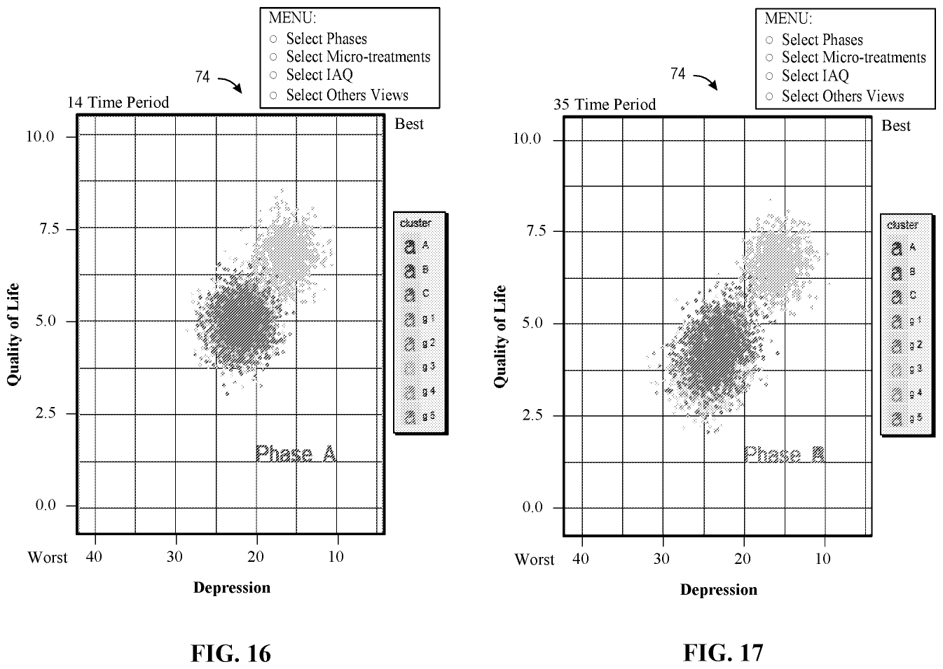

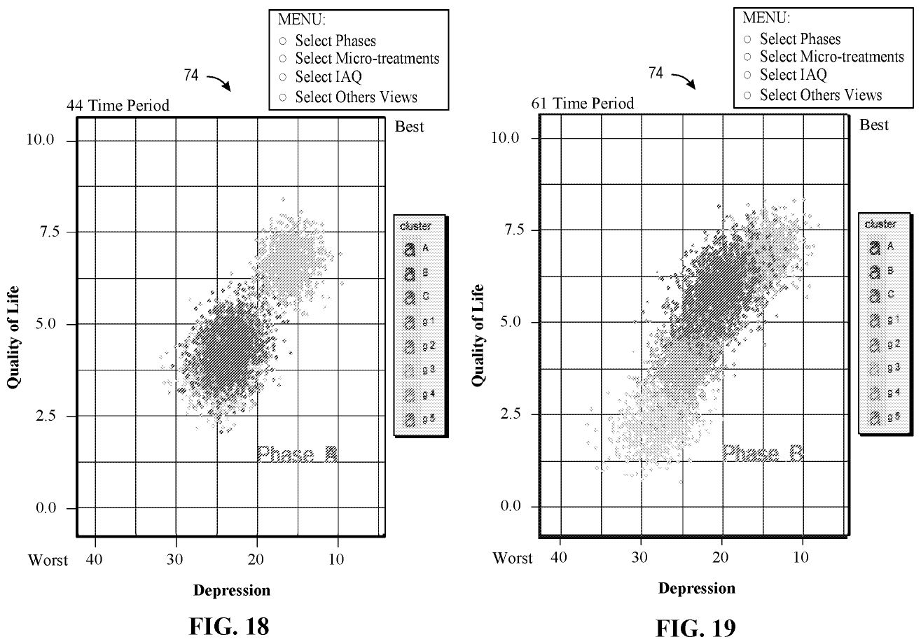

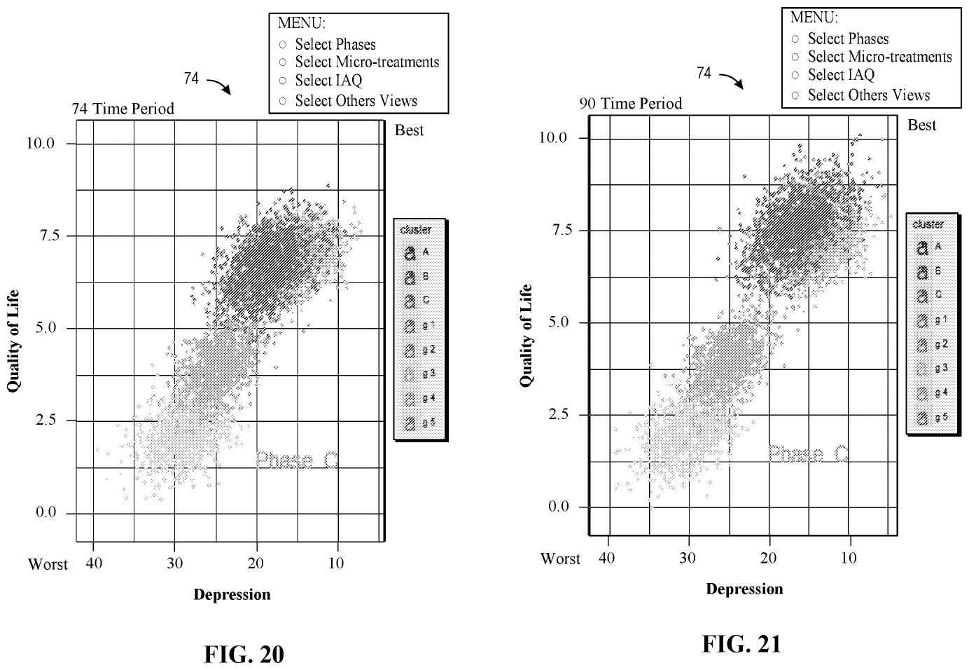

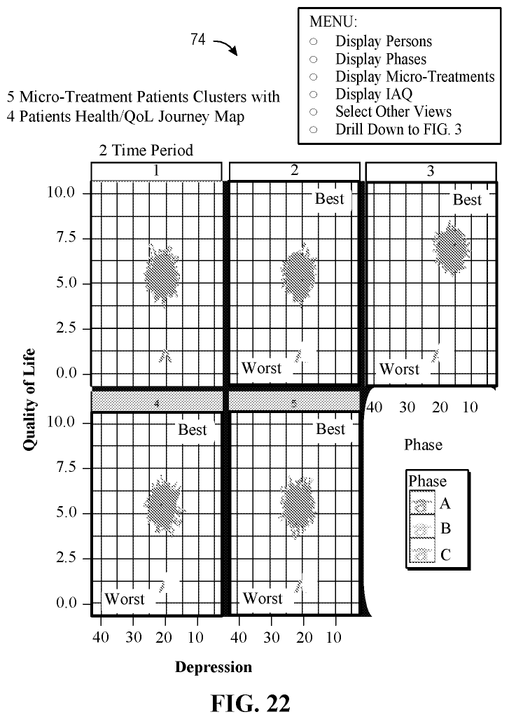

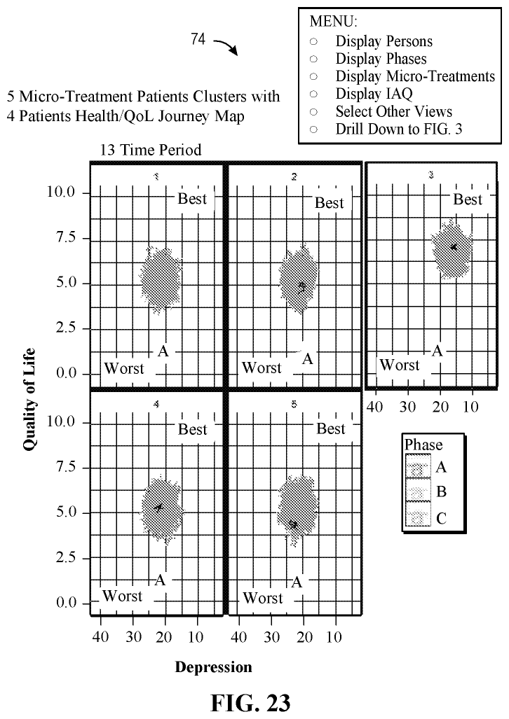

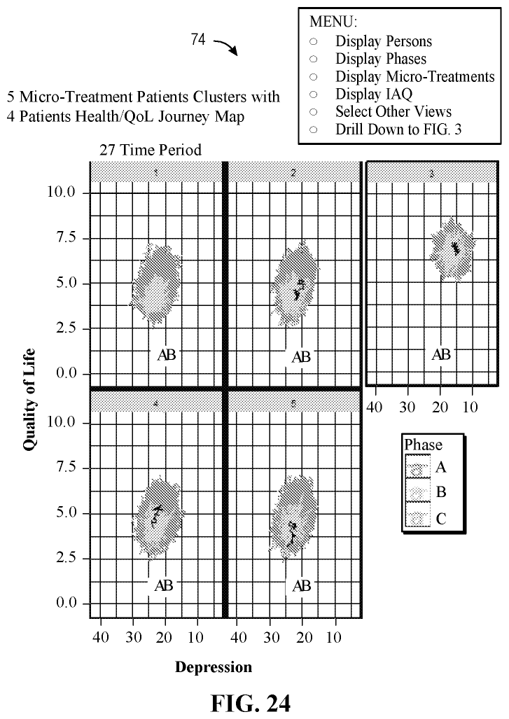

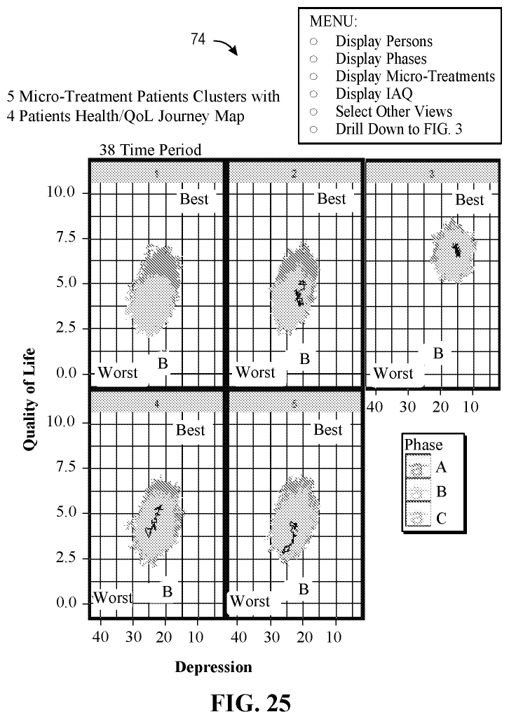

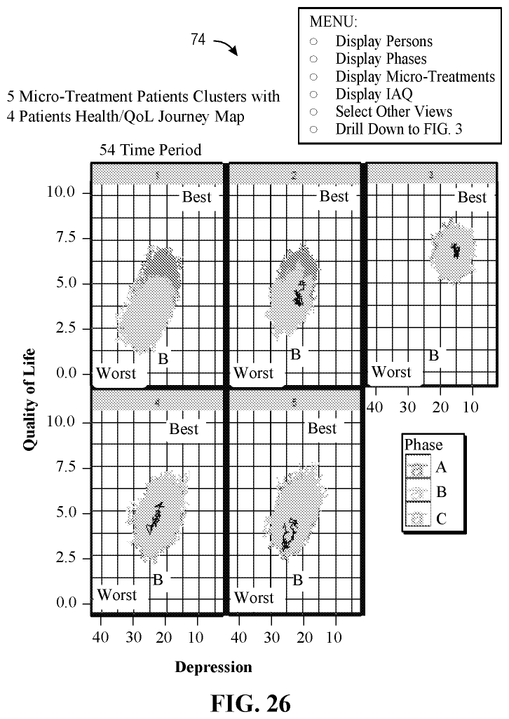

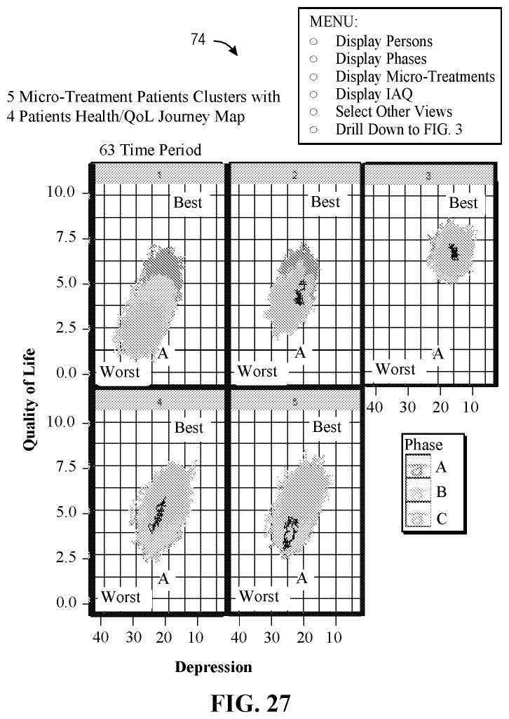

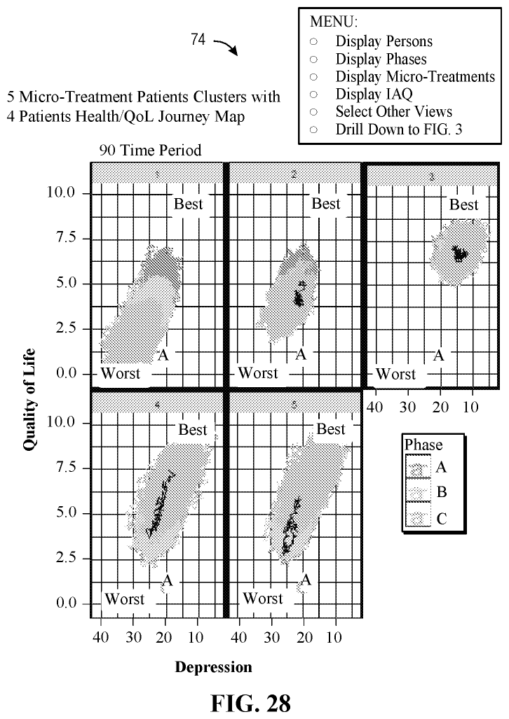

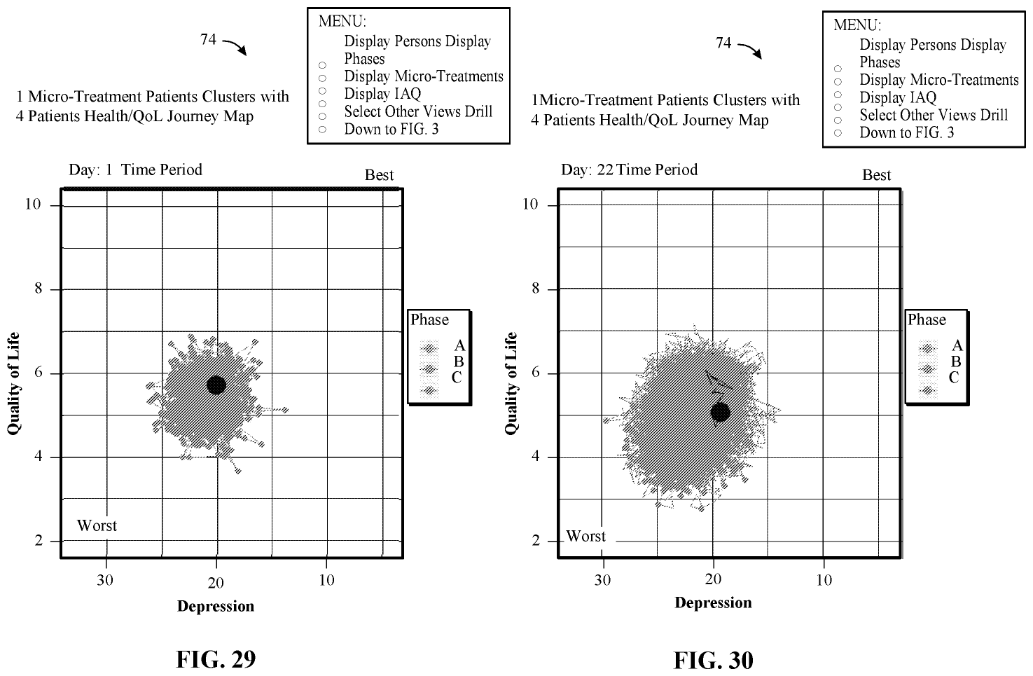

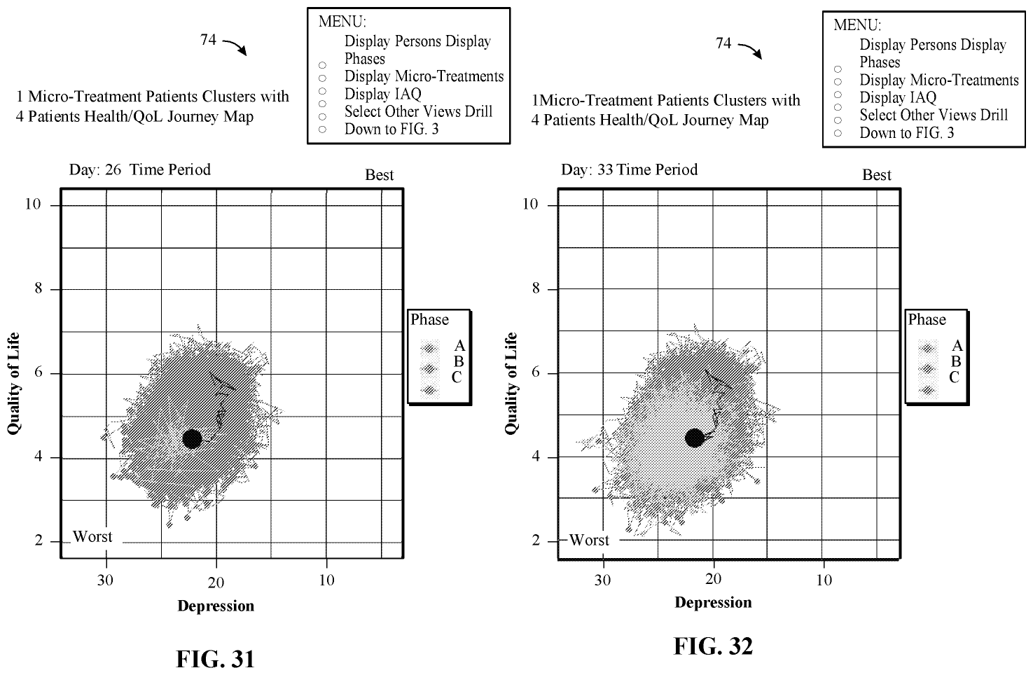

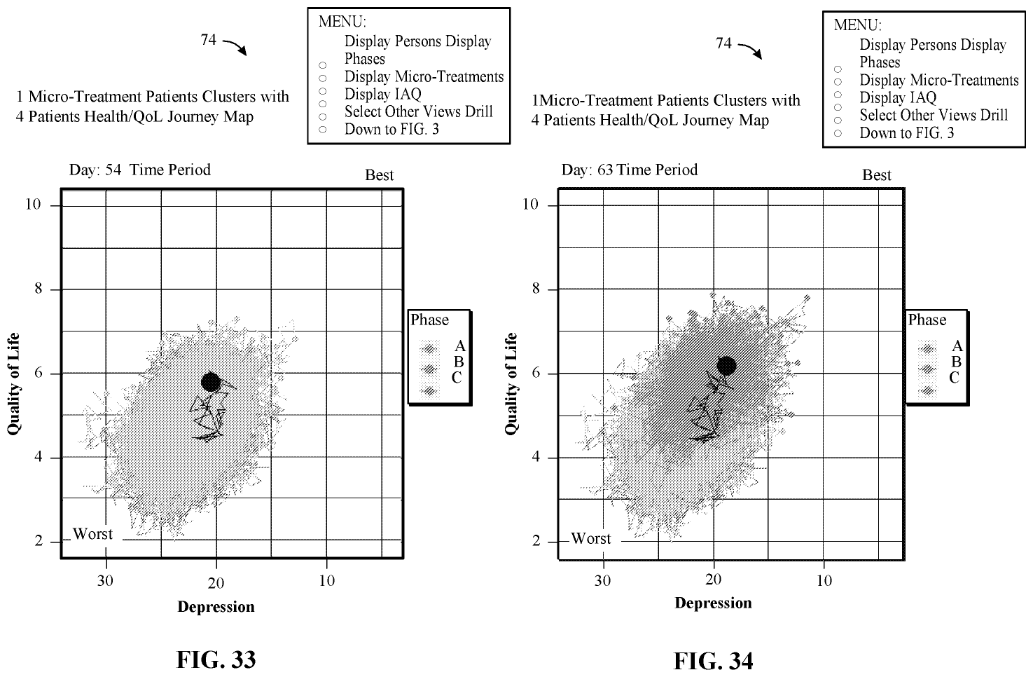

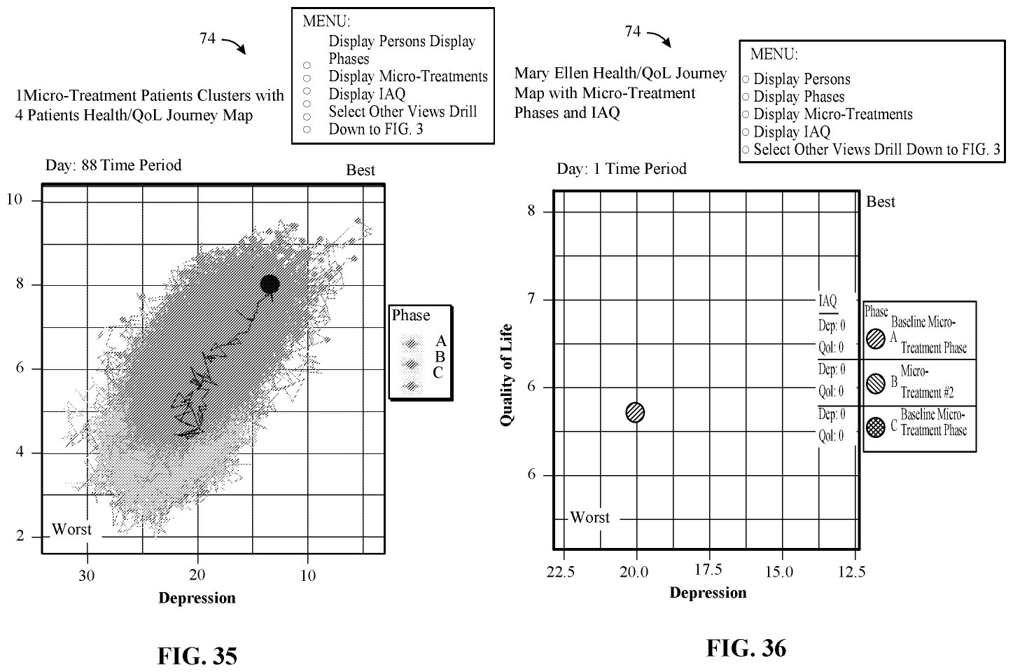

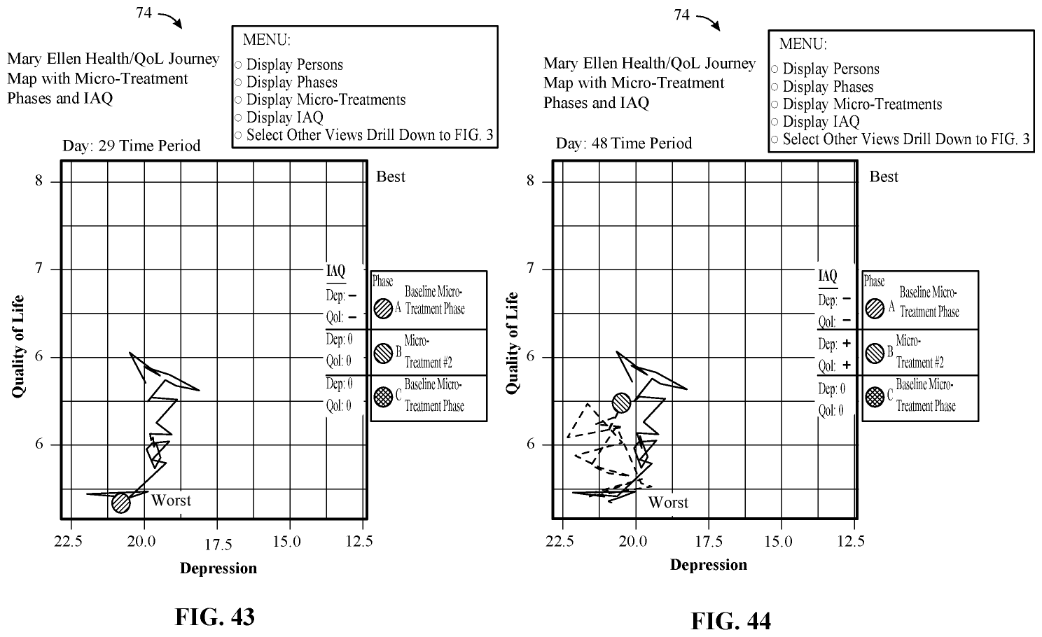

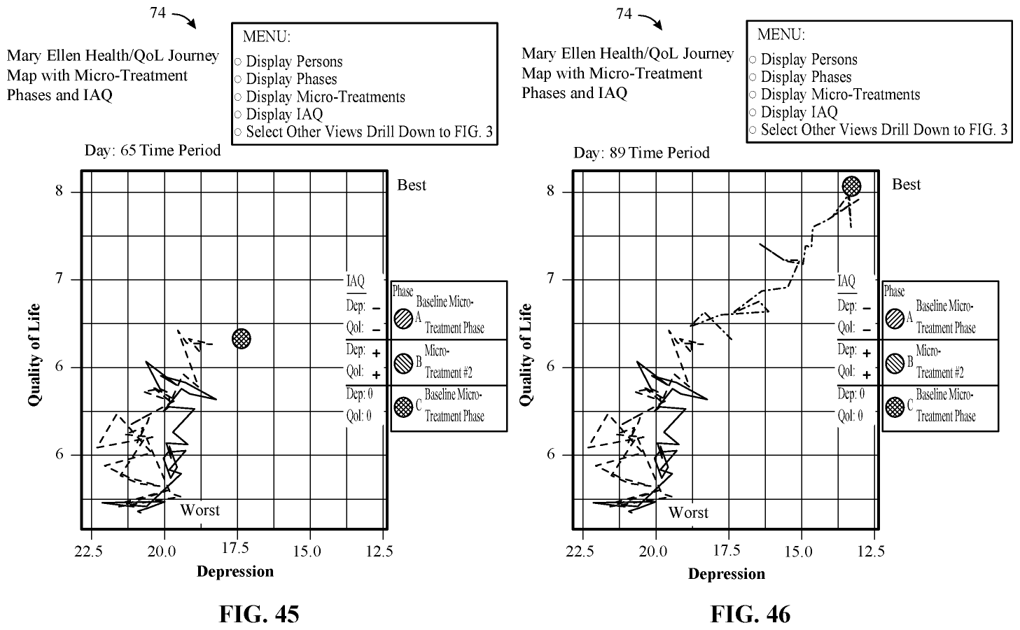

The treatment system 100 allows a healthcare team, consisting of a patient and the healthcare providers, to achieve personalized treatment outcomes, with high confidence, while significantly reducing the burdens associated with treatment of the individual using individual science (N-of-1) alone. The treatment system 100 frequently captures data from a patient N in real-time, and the data is presented on a dashboard 40, i.e., a digital dashboard, as different treatment segments or “phases”, e.g., micro-treatments 42, to determine which, if any, treatment interventions may be required. Referring to FIGS. 3A-3C, exemplary Phases 52 may include Phase A, Phase B, and Phase C are shown on a personal treatment plan for a single patient. The treatment plan for Phase A is different from Phase B and Phase C, that outcome results 44 are shown on an X-Y graph in terms of depression 46 and a quality of life (QoL) 48 (on a Y-axis) along intervals of time (on an X-axis). The visualization of the results of the different treatment interventions assist healthcare providers with providing better informed, and more efficient, treatment decisions for the patient. In one non-limiting example, the treatment system 100 may capture data for a particular micro-treatment from the patient daily, with the segment of the micro-treatment lasting for one-month. It should be appreciated, however, that the Phases 52 are not limited to A, B, and C, as any number of Phases 52 A-X^{th} may be included.

Patients N are medical patients, individual humans, or other complex systems, like but not limited to animals, plants, artificial intelligence devices, weather, etc. Healthcare providers may include, but should not be limited to, physicians, medical physicians, nurses, psychologists, pharmacists, physician assistants, or other professional care providers or complex system specialists, scientists, self-scientists, and the like. The healthcare provides may also include the actual patient and/or the caregiver to the patient, due to the intimate knowledge associated with the conditions being treated and their effects. The treatment team may also include home care providers, such as nurses, family members, friends, and the like who may assist the patient N with compliance with their treatments and/or data entry.

The treatment system 100 includes a data server 10 in communication, via a network 76, with a patient user device 70, a healthcare provider user device 71 and the like. In the example shown, the treatment system 100 can include a wearable device 73 in communication, via the network 76, with the data server 10. The example shown in FIG. 1 is non-limiting, such that treatment system 100 can be configured such that the data server 10 can include other user devices, and other devices suitable for monitoring, measuring, and/or recording physiological data, psychophysiological data, environmental data, and/or geographic attributes, relevant to the patient, in real time, to provide a patient's digital health knowledge. Alternatively, in another non-limiting example, the treatment system 73 may be encapsulated within the wearable device 73 as a standalone system.

With many ailments, relief and/or a cure may be provided to a patient N through treatments that may include, but should not be limited to, the adoption of a particular diet, the adoption of a particular lifestyle, taking a prescribed medication, and/or the like. The advent of personal computing devices, i.e., user devices 70, 71, and wearable devices 73 have improved the ability of patients N and/or the patient's caregivers to self-monitor the effectiveness (or lack of effectiveness) of a particular treatment of the ailment on the patient N, or lack of adherence to the particular treatment by the patient N, when not in the continued presence of the healthcare provider D. However, the concept of self-monitoring faces significant challenges because self-monitoring, by itself, does not often lead to a sustained behavior change and self-monitoring requires a behavior to be operationalized and recorded for analysis, presentation, and interpretation at a later point in time. This historically has been a labor-intensive prospect for the person doing the self-observation (e.g., the patient N and/or the non-professional or professional caregiver) and adherence to good data collection can be difficult. For example, Alzheimer's Disease patients typically require significant support with medication monitoring due to confusion and forgetfulness, associated with cognitive decline.

Digitally enabled mobile tracking applications (typically embodied in wearable devices 73 or other patient user devices 70), can help solve both of the challenges otherwise faced by self-monitoring, by tracking and recording digital health knowledge relating to the patient N being treated. When designed properly, mobile tracking applications associated with such devices 70, 73 can be pre-programmed with structure to alert the patient N or the caregiver about activities to be performed, operationalization goals, and related target behaviors (i.e. sub-goals), data analysis, recording of the data, and presentation of the collected data. Operationalization is the process of defining the measurement of a phenomenon that is not directly measurable, though its existence is indicated by other phenomena. By way of a non-limiting example, in medicine, a health phenomenon might be operationalized by one or more indicators like a body mass index, amount of alcoholic beverages consumed per day, the amount of exercise attained per day, the amount of sleep per night, happiness on a particular day, perception of a quality of life on a particular day, and the like. The health of the patient N may be monitored and measured by setting one or more operationalization goals, such as requiring at least 8 hours of sleep per night, walking one mile per day, drinking one glass of wine per day, and the like. In doing so, a relationship between the operationalization goals and one or more health outcomes may be observed and recorded, such as, the patient's happiness each day, the patient's perception of a quality of life, heart rate, and the like.

However, it should be appreciated that treatments for patients N with many ailments are not universal. For example, with respect to Alzheimer's Disease, the current medications provide meaningful relief to less than five percent of patients. Some studies have suggested that some patients receive benefit from merely taking a placebo, while other patients receive benefit from a combination of the medication and receiving a certain amount of exercise each day or other non-medication treatments. However, as already discussed, the ability to determine which treatment, or combination of treatments, would work best for a specific patient N through only the application of N-of-1 science is typically time consuming.

In comparison, the treatment system 100 of FIG. 1 is configured to combine existing, validated group/aggregated data (e.g., clinical guidelines, evidence-based treatment goals, etc.) with individual patient N data points to place the individual patient's N response in a context of within the individual comparison (i.e., N-of-1 patient N level change across two or more conditions) and between the individual and population based comparator (e.g., guidelines, goals, etc.). The treatment system 100 then aggregates response data 22 from the patient N up in a building series of N-of-1 replications in order to identify unique patient groups, with unique outcome pathways. The identification of unique patient groups is accomplished via the application of a combination of inductive, abductive, and deductive logic to place a given patient N within a segment. A segment is defined as the use of any number of techniques intended to create subgroups based on optimized homogeneity within a segment and optimized heterogeneity between segments. A segment can be also defined with inclusion or exclusion attributes. Once identified, the treatment system 100 is configured to track that given patient N relative to their assigned segment, and based on their time-series response data 22. The treatment system 100 is further configured to track the progress of the patient N, relative to each of the identified segments, thereby determining the individual change of the patient N, relative to more positive/negative segment pathways. As such, by combining self-monitoring of the patient N through the incorporation of the patient user devices 70, wearable devices 73, sensors 75, healthcare provider user devices 71, and the like, by implementing the treatment process 200 (FIG. 2), the treatment system 100 allows for real-time individual patient N monitoring and evaluation of treatment response, over time, to rigorously evaluate treatment effectiveness.

In one embodiment, the treatment process is configured to evaluate patient N response data 22, e.g., time-series data, gathered at a minimum of two points in time, at the level of the individual unit (e.g., N-of-1 evaluation using inductive reasoning for individual patient N level time-series response data 22). Such an evaluation will be able to determine whether there has been a meaningful change between two or more evaluative conditions (as will be explained in more detail below).

In another embodiment, the treatment process may be configured to aggregate the individual patient's N N-of-1 evaluations (i.e., replication of conditions and the outcomes), based on deductive reasoning for the determination of collective outcomes, based on configurable thresholds for sufficient/significant replications to determine “collective” outcomes of the N-of-1 replications.

Additionally, the treatment process may be configured to track time-series response data 22 recorded in the data store structure 18, collective on an individual patient N, relative to a comparator data point/path, over time (e.g., nature or a disease or treatment, EBM guideline, personal treatment plan or goal, and the like). The time series-response data 22 includes a person-level data signature.

Therefore, the treatment process 200 applied by the treatment system 100 is configured to provide the individual application of established group data, in combination with the individual patient N-of-1 evaluations, relative to established group data. The established group data may include, but should not be limited to, best practices, guidelines, clinical trials, etc. The N-of-1 replications associated with the individual application of established group data is aggregated and inductively evaluated in order to identify an outcome pathway (i.e., segment pathway development) relative to established deductively reasoned group data. The treatment process is further configured to identify and evaluate a combined personalized care pathway for a patient N, based on a combination of the group data and individual treatment response. It should be appreciated that the system 100 may be configured to record the outcomes to further grow and refine the established group data.

As shown in FIG. 1, the data server 10 of the treatment system 100 includes a central processing unit (CPU) 12, which may also be referred to herein as a processor 12. The data server 10 can employ any of a number of computer operating systems, including, but not limited to, versions and/or varieties of the Microsoft Windows® operating system, the iOS by Apple Computer, Inc., Android by Google, Inc., the Unix operating system (e.g., the Solaris® operating system distributed by Sun Microsystems of Menlo Park, Calif.), the AIX UNIX operating system distributed by International Business Machines (IBM) of Armonk, N.Y., and the Linux operating system or any other CPU operating system. The processor 12 receives instructions from a memory, such as memory 14, a computer-readable medium, etc., and executes these instructions, thereby performing one or more processes, including one or more of the processes described herein. The computer-executable instructions may be compiled or interpreted from computer programs created using a variety of programming languages and/or technologies, including, without limitation, and either alone or in combination, Java™, C, C++, Visual Basic, Java Script, Perl, html, etc. Such instructions and other data may be stored and transmitted using a variety of computer-readable media. By way of non-limiting example, the memory 14 of the CM server 10 can include Read Only Memory (ROM), Random Access Memory (RAM), electrically-erasable programmable read only memory (EEPROM), non-volatile memory, etc., i.e., non-transient/tangible machine memory of a size and speed sufficient for storing a data store 18 including a data structure 26, algorithms 20, response data 22, and one or more database management applications 24, which can include, for example, a relational database management system (RDBMS), a non-relational database management system, and the like. The data structure 26 can include one or more databases, data tables, arrays, links, pointers, etc. for storing and manipulating the response data 22. The response data 22 can include, by way of non-limiting example, patient profile data, patient raw data, pre-processed time series data, patient micro-treatment confidence score data, micro-treatment suggestion data, etc. for one or more patients N, as required to allow the treatment system 100 to perform the treatment processes 200 described herein. The memory 14 is of a size and speed sufficient for manipulating the data structure 26, for executing algorithms 20 and/or applications 24, and to execute instructions as required to perform the treatment processes 200 described herein. The data server 10 includes a communication interface 16, which in an illustrative example can be configured as a modem, browser, or similar means suitable for accessing a network 76. In one example, the network 76 provides data communications that may include, but should not be limited to, the internet, cellular phone data networks, satellite data networks, etc.

With continued reference to FIG. 2, the data server 10 can include various modules, such as a data module 28, an evaluation module 30, an aggregation module 32, a display module 34, a suggestions module 36, a micro-treatment module 38, and the like, described in further detail herein. The various modules 28, 30, 32, 34, 36, 38 can process, link, and analyze different types of data, generate static displays, generate animated displays, generate reports, generate models, recommend micro-treatments, etc., using algorithms 20 and/or instructions which may be stored within the different modules 28, 30, 32, 34, 36, 38, in the data store 18, and/or in one or more of the user devices 70, 71, wearable devices 73, and the like, in communication with the data server 10.

The algorithms 20 can include, by way of a non-limiting example, one or more algorithms 20 for organizing time series data from a patient for optimal processing or standardized presentation, one or more algorithms 20 for aggregation of N-of-1 replications, one or more algorithms 20 for generating one or more types of displays on display screens (input/output interfaces 74) of one or more user devices 70, 71 associated with the time-series data from the patient N, one or more algorithms 20 for generating one or more micro-treatment recommendations, one or more algorithms 20 for prescribing a micro-treatment to the patient N, as described in further detail herein. The examples describing the data server 10 provided herein are illustrative and non-limiting. For example, it would be understood that the functions of the data server 10 may be provided by a single server, or may be distributed among multiple servers, including third party servers, and that the data within the system 100 may be distributed among multiple data stores, including data stores accessible by the data server 10 via the network 76. For example, it would be understood that the plurality of modules shown in FIG. 1, and the distribution of functions among the various modules 28, 30, 32, 34, 36, 38 described herein, is for illustrative purposes, and the module functions as described herein may be provided by a single module, distributed among several modules, performed by modules distributed among multiple servers, including modules distributed on multiple servers accessible by the data server 10 via the network 76, and/or performed by the data server 10.

With continued reference to FIG. 1, as already discussed, the treatment system 100 may include one or more user devices 70, 71 (i.e., one or more patient user devices 70, one or more healthcare provider user devices 71, and the like), which can be in communication with one or more data servers 10, via the network 76. The user devices 70, 71 each include a memory 66, a central processing unit (CPU) 68, which can also be referred to herein as a processor 68, a communication interface 72, and one or more input/output interfaces 74. The user devices 70, 71 may be a computing device such as a mobile phone, a personal digital assistant (PDA), a handheld or portable device (iPhone®, Blackberry®, etc.), a wearable device 73 (i.e., a Fitbit®, Garmin®, smartwatch, etc.), a notebook computer, a laptop computer, a personal computer, a tablet, a note pad, or other user device configured for mobile communications, including communication with the network 76, with other user devices, the data server, and the like.

It should be appreciated that one or more of these patient user devices 70 may be in communication with one or more electronic and/or MEMS sensors, actuators, and/or other computing devices configured to capture digital health knowledge data from the patient N. These may be wearable devices that are configured to provide digital health knowledge and/or are therapeutic. The sensors 75 are used to measure certain parameters of the human body, either externally or internally. Examples include, but should not be limited to, measuring the heartbeat, body temperature, or recording a prolonged electrocardiogram (ECG). By way of a non-limiting example, these sensors 75 may be incorporated into one or more wearable sensors 75 (e.g., earring, tattoo, smart textiles, wristbands, glasses, ring, etc.), implantable devices (e.g., pacemaker, etc.), smart pills, injectable devices, ingestible devices, etc.

The actuators may be configured to take one or more specific actions, in response to data received from the sensors 75, or through interaction with the patient N, caregiver, healthcare provider, and the like. By way of a non-limiting example, the actuator may be equipped with a built-in reservoir and pump that administers the correct dose of insulin to the patient N, based on the glucose level measurements. Interaction with the patient N may be regulated by a personal device, e.g. the user device 70, the wearable device 73, and the like.

The user device 70, 71 may be configured to communicate with the network 76 through the communication interface 72, which may be a modem, mobile browser, wireless internet browser or similar means suitable for accessing the network 76. The memory 66 of the user device can include, by way of example, Read Only Memory (ROM), Random Access Memory (RAM), electrically-erasable programmable read only memory (EEPROM), etc., i.e., non-transient/tangible machine memory of a size and speed sufficient for executing one or more data management applications which may be activated on the user device 70. The input/output interfaces 74 of the user device 70 can include, by way of example, one or more of a keypad, a display, a touch screen, one or more graphical user interfaces (GUIs), a camera, an audio recorder, a bar code reader, an image scanner, an optical character recognition (OCR) interface, a biometric interface, an electronic signature interface, etc. input, display, and/or output, for example, data as required to perform elements of the treatment process 200. The example shown in FIG. 2 is non-limiting, such that it would be understood that the treatment system 100 can include multiple patient user devices 70, multiple healthcare provider user devices 71, multiple wearable devices 73, user devices associated with a caregiver, and the like, each in communication with the network 76. For example, the treatment system 100 can include patient user devices 70 used by one or more patients and one or more healthcare provider user devices 71 used by one or more healthcare providers D in the treatment of one or more patients N, as described in further detail herein.

Referring to the data server 10 shown in FIG. 1, in one example, the data module 28 can be configured to receive, record, and organize data submitted to the treatment system 100 by the patient and/or a caregiver of the patient associated with the act of self-monitoring, via one or more user devices 70. The data module 28 can include algorithms 20 for parsing, formatting, and recording data associated with the patient.

Response data 22 recorded from the act of self-monitoring can be made more accurate if data error associated with behavioral architecture of the wearable device 73 is dealt with. In one embodiment, the patient N response data 22 is automatically collected in real-time. Potential error is introduced into the data when the patient's recall is required, and this error may be eliminated or significantly reduced by virtue of automatic data collection (or by virtue of required minimal engagement), in real-time (i.e., in the moment, or near real time) data collection. By automating data collection, to the degree that the patient N need not engage in a specific behavior to actually initiate recordation of the data, the data fidelity is enhanced (e.g., steps, circadian rhythm, heart rate, amount of ultraviolet light (UV) light exposure, medication taking, etc.). The automatic data collection should also include the time the data was collected to provide time series data. If the patient N does need to take action (e.g., enter a number, press a button, take a medication, etc.) to initiate the recordation of response data 22, the recording should occur contiguously with the act, in real-time (and have the time recorded or time stamped) to provide time series data. In some instances, the time recordation is the time and date, in other instances, the time recordation may be more general, such as a date, a month, and the like. In other instances, the data recordation may also include a geographic location, temperature, weather conditions, and the like.

Since the act of simply presenting the collected data from the wearable device 73 to the patient, no matter how clearly and creatively presented, is typically insufficient for enacting a sustaining change, a display on the wearable device 73 and/or user device 70 may be configured to display a direction to the patient N regarding insights about triggers of behaviors so as to assist the patient N with distinguishing between internal and external triggers. The triggers may include, thoughts, feelings, actions, and the like, including somatic behaviors (e.g., pain, palpitations, stomach pain, diarrhea, etc.). These triggers are temporal connections (i.e., correlations, prediction models, etc.) between antecedent and behavior.

Changing of relevant metrics for a patient N may be built around frequency, intensity, duration, and course (trend), as relevant to a target behavior(s). Further, internal triggers and external triggers may be distinguished. The data associated with contiguous relationships (i.e., high cause/effect probability) among variables may be arranged and presented to the patient N in meaningful ways. To do so, an understanding is made regarding patient's level of motivation (e.g., 2 to 6 levels) and confidence (e.g., high or low). All of the latest data status and trend implications for the patient may be assessed. Further, the display on the display device 70 and/or the wearable device 73 may encourage the patient to select self-determined experiments from a library of treatment options that are most suitable for a main health goal and/or a main quality of life goal (e.g., the ability to optimize up to five total variables outcomes in a future segment).

In one embodiment, an expert system may be provided to balance (i.e., score) the treatment options available in the library, suggestions module 36, to provide a library of the best or preferred treatment options. The library of treatment options may be used based on a relationship to the patient, e.g., level of motivation, level of confidence, set-limit of presented options (e.g., 1 to 5), best self-experiment segment micro-treatment recommendation design (AB, ABAAB, ABCA, multiple baseline, others with time duration of each segment), and the like which when aggregated would get smarter via volume of N-of-1 replications. The library of treatment options may include best or preferred group average science treatments, N-of-1 science input from the patient, N-of-1 science input from friends or others within the population, ideas/theories, reference data, and the like.

The best or preferred group average science treatments are cited science studies that may include, but should not be limited to, pharmaceuticals with FDA approval and non-pharmaceutical. The best or preferred group average science treatments may be based off of established group data (e.g., best practices, guidelines, clinical trials, etc.). The N-of-1 science from the patient or the friends/crowds or others within the population may be categorized as weak to none (e.g., under 49%), some (e.g., 50%-69%), moderate (e.g., 70%-89%), or strong (90%-100%) or the like. The ideas/theories may initially have an unknown value, or may have a possible value with some supporting theory. The reference data may include links to science articles and the like to provide support and general how-to information.

Functional components of a behavioral analytical architecture, may include, but should not be limited to, triggers, actions, instrumental behavior, biological behavior, cognitive behavior consequences, psycho-social behavior, exercise behavior, diet behavior, and the like. The triggers (i.e., antecedents, stimuli, etc.) are any perceptible cue occurring temporally prior to a target (i.e., behavioral, biological, cognitive/emotional) and often “triggers” the target. Triggers can be external to the observed (in the environment) or internal to the observed (subjective states). Actions (i.e., overt acts, cognitive, emotional or biological actions) are a change in the status of the observed in the target in response to contextual factors (internal and external to the observed). Instrument behavior is any overt act made by the observed (e.g., smoke a cigarette, run, take a medication, etc.).

Item response theory (IRT) (i.e., latent trait theory, strong true score theory, modern mental test theory, and the like) is a model for the design, analysis, and scoring of tests, questionnaires, surveys etc., based on the relationship between and individuals' response to a given test item and their score or performance on an overall measure. IRT does necessarily treat each item with equal weight, but rather uses the weight of each item (i.e., the item characteristic curves, or ICCs) as information to be incorporated in scaling items. IRT can be used to measure human behavior in online social networks whereby the views expressed by different people can be aggregated and studied.

Biological behavior is any physiological or psychophysiological change in response to the status of the biological functioning of the observed (e.g. heart rate, BMI, A1C, sleep architecture, etc.). Cognitive behavior is the processes of knowing, including attending, remembering, reasoning or others; also the content of the processes, such as but not limited to concepts and memories. This includes but is not limited to interpretations of cause and effect relationships, motivations, self-perceptions, and moral reasoning. Consequences may include changes in the internal and/or external environment of the observed that meaningfully follows an act.

The treatment system 100 is configured to provide a display 40 and interpretation relating to a patient's progress, periodically assesses goals and motivations to recommend goal changes (up or down), compare an opted-in group's progress, compare known population-based progress, compare a patient's data to a friend's progress who has “opted-in” and/or others, compare crowd progress, and the like. This type of display and interpretation may make is easy to spot or see trends; make it easy to keep on a treatment journey if good value is being realized by the patient, make it easy to switch to a different journey if poor value is being realized by the patient or no value is determined.

The treatment system 100 may provide many features, including, but not limited to, initial onboarding, an express lane startup, a major infrequent stressor event recording, multi-channel support structure, and the like. The initial onboarding is configured to allow easy and progressive surveying that is flexible to gather up front, first time data. Each use case will be prioritized differently, and few people will have to complete everything in the survey initially. The express lane startup is configured to provide the ability to pull information from a patient's electronic health records (EHR) and/or to exercise device data. The emergency off ramp provides a special triage for a patient's emergency risks, e.g., no pulse, falls, left a geo-fence area, fire at location, etc. The major infrequent stressor event recording is configured to provide a simple and easy way to log births, deaths, job end, job start, theft, accidents, etc. Further, the multi-channel support structure is configured to provide support outside of an application integrated communication, uses an integrated communication application to support streams of automated conversations with text, video, audio, tele-specialists, email (with web links), and the like. Uses “double helix” (friend/family/community) connections to help support the user.

An application program interface (API) may be provided to “strategic partners”, such as support specialists in disease, disorder, and health science with better experience, behavior science, social support, care team communications, and artificial intelligence (AI) expert decision support, N-of-1 individual science analytics, and small and large group analytics. Providing the API to these strategic partners, allows for the leverage of specialist user interaction with specialized knowledge, special outlier cases, exceptions, and gamification knowledge.

Wearable device 73 and sensor API integration may also be configured to be friendly to top devices and sensors 75, while providing flexibility to add new devices and sensors 75, over time. The FDA and various medical standards of recording an identification (ID) of the wearable device 73, along with its calibration steps and history, may be linked to a period of patient data. There is a level of accuracy associated with the ability of the wearable device 73 to learn and distinguish between and noise. As such, the wearable device 73 may be configured to only record and/or transmit a patient data feed associated with signals, while ignoring noise.

The system 100 is configured to receive, process, and record data feeds from the patient. A patient data feed may be an ongoing stream of structured and unstructured data that provides updates of current information (i.e., time series) from one or more sources. “Big” data (i.e. data that is complicated to store, organize, evaluate, and present in a context that requires the consumption of large volumes of data (data that exceeds natural human capacity), analysis of that data using complicated mathematical processes with significant speed such that findings can be meaningfully displayed back to an end user device for timely decision making.

Several non-limiting examples of big data feeds provided by the treatment system 100 may include, but should not be limited to, geolocation and weather by the hour with the ability to roll up into summaries by day linked to users and their location; drug data linked to side effects and risks (allows the spotting of N-of-1 early issues earlier); other known science databases, such as, known EMI maps, earthquake maps, pollution maps, etc.; digital map API (e.g., Google, NavTec, and the like) to assist with finding healthy activities or food (e.g., FitCare); healthcare portal partners work with patient and/or mainstream employee health portals (e.g., major EHR like EPIC) to share data and ultimately increase the value of the portals.

The treatment system 100 provides the treatment process 200 shown in FIG. 2. The treatment system 100 combines inductive, abductive, and deductive logical inference, and related analytical methods to evaluate and analyze a plurality of time series data and/or repeated measures data (i.e., continuously collected and evaluated over a specified time period) at the individual unit N (e.g., single patient, single complex system, or N-of-1), based on the assignment of a discrete micro-treatment at each segment. A micro-treatment 42, corresponding to a Phase 52, may be defined as a blend of a specific dosed medication or non-drug treatment or any behavior, life style, environment or system change or combination (or any other prescribed, defined, known, or unknown variable) for a certain period of time (relative to a baseline or other comparative state). The system 100 evaluates a plurality of N-of-1 “segmented” evaluation methods that compare change in the patient data between two (or more) distinctly characterized segments, i.e., discrete micro-treatments administered during fixed time periods, with at least two measures per segment. A segment has one or more dependent variables and one or more independent variables, measured across time.

There are many N-of-1 analysis tools and methods. It should be appreciated that any one of these N-of-1 analysis tools, in combination with a design of an individual system for experimenting with changes between segments, by varying one or more independent variables in order to measure the effect on one or more dependent variables. The sensors 75, wearable devices 73, data server 10, user devices 70, 71 are configured to gather response data 22 and calculate a level of change as measured against normal or well-researched ranges (GAS) and to calculate a level of association that the independent variable cause (or did not cause) a change to the dependent variable. The level of change and level of association can be shared via wired or wireless electronic communication and with or without a computer server to support additional analytics and to provide summary visualization to one or more users, including the patient N, the doctor D, the caregivers, and the like. In one non-limiting example, with reference to FIG. 2, the independent variable may be the elements of the micro-treatment prescribed (assigned) to the patient N. The elements (independent variables) of the micro-treatment may include a regimented amount of exercise, a specific diet, and a specific medication. With continuing reference to FIG. 3, the dependent variables may be measured in terms of depression and a quality of life. For each of these, an IAQ score 22D is assigned and may displayed on the display screen in terms of the overall treatment segment, and at each time unit (e.g., daily).

With reference between FIG. 1 and FIG. 2, the system 100 is configured to calculate and generate a metric, such as a measure of change in the patient data and/or a confidence score (i.e., “IndividuALLytics Quotient” (IAQ) score) from one segment to another segment, in terms of valance (i.e. positive/negative impact), direction (up/down), and effect size and/or calculated standardized measures and/or a relative level of micro-treatment compliance 46 during each segment and/or confidence intervals for balancing Type I and Type II errors. In statistical hypothesis testing, a Type I error is known as a “false positive” finding, while a Type II error is known as a “false negative” finding. A Type I error is to falsely infer the existence of some relationship that is not there, while a Type II error is to falsely infer the absence of some relationship that is there. The IAQ score provides a user of the treatment system 100 with a score that represents the statistical confidence associated of the effect of a micro-treatment on a patient N for a particular segmented time-period (e.g., one-month) and/or at a particular interval (e.g., one day). With specific reference to FIG. 3, the IAQ score may be graphically represented in terms of “++”, “+”, “0”, “−”, and “−−”, where an IAQ score of “++” may indicate a confidence level of greater than e.g., 80% (the specific confidence percentage would be configurable based on the end user's preferred balance of Type I and Type II error) that the micro-treatment was effective, an IAQ score of “−−” may indicate a confidence level of greater than or equal to 80% that the micro-treatment was ineffective and provided a negative impact on the patient N, an IAQ score of “0” may indicate a confidence level of under 50% that the micro-treatment provided no impact on the patient N. Likewise, an IAQ score of “+” and “−” may indicate a predefined confidence level of between 50% and the 79.9% that the micro-treatment likely had some impact on the patient N either positively or negatively. It should be appreciated, however, that the disclosure is not limited to having these confidence levels, only five levels of IAQ scores, and/or having IAQ scores represented in the form of “+”, “−”, and “0”, as any other suitable indicator of a confidence score may be graphically represented on the display of the user device 70, 71. Such a graphical representation allows a healthcare provider (such as a doctor D), the patient N, the caregiver(s), and the like, to quickly determine the effectiveness/ineffectiveness of a particular micro-treatment and/or the level of decision confidence.

With reference to FIGS. 5-45, the system 100 may also present evaluated time series data 82 in an animated fashion on a graphical user interface (GUI), at each of the level of the individual unit N (single patient), the defined groups of the individual units N, and the overall population. The determination of defined groups of the individual units, when combined with the graphical representation of such associations, based on IAQ scores, provides a graphical representation of treatment effects that allows a user to quickly and easily make a visual determination of the effectiveness/ineffectiveness of a particular treatment. A healthcare provider or patient can use menus 60 to further evaluate level of confidence and effectiveness/ineffectiveness of micro-treatments for one patient, several patients, many patients or all patients. The menus can include but not limited to select persons, display segments, display micro-treatments, display IAQs, select other views, and drill down to FIG. 3. An example treatment process 200 will now be described with reference to FIG. 2.

The treatment process 200 according to an example embodiment commences at step 202, wherein a patient profile 22A (FIG. 4) regarding a patient N is created and recorded in the database 18 to become part of the response data 22. The patient profile 22A may include, but should not be limited to, a patient's name, age, current diagnosis, current prescribed medications, past surgeries, mental health status, hospitalizations, genetic profile, allergies, health goals, life goals, family care givers, family medical history, medical record identifier, anonymous record identifier, and the like. The process 200 then proceeds to step 204.

At step 204, the server 10 receives raw response data 22B (FIG. 4) from the patient N. The raw response data 22B may be received from one or more patient user devices 70, wearable devices 73, sensors 75, healthcare provider user devices 71, and the like, via the network 76. The raw response data 22B corresponds to the effects on the patient N over a time-period (i.e., treatment segment) for a discrete micro-treatment. The raw response data 22B may be recorded in the database 18 at step 206. The process 200 then proceeds to step 208.

At step 208, one or more algorithms 20 may be initiated by the processor 12 to pre-process the raw response data 22B to provide a time-series data set 22C (FIG. 4). In pre-processing the raw response data 22B in order to provide the time-series data set 22C, the algorithm 20 may be configured to standardize or normalize the raw response data 22B and/or identify and correct for any missing data within the raw response data 22B. In doing so, the algorithm 20 may use any of a variety of known techniques, based on optimal methods for managing data gaps, such as auto-correlation, mean substitution, max value, and the like. The time-series data set 22C may be recorded in the database 18 at step 210. The process 200 then proceeds to step 212.

The process 200 entails the optional step 212 of incrementing a counter C. The process 200 then proceeds to step 214.

Optional step 214 entails determining whether a predefined number of treatment segments (C=CAL) have been pre-processed and recorded as a time-series data set 22C in the database 18. For instance, the processor 12 may increment a counter (C) following the completion the recordation of the time-series data set 22C in the database 18 at step 210. It should be appreciated, however, that the process 200 may be configured to increment the counter (C) following any of the data steps 204, 206, 208, 210, without departing from the scope of the disclosure. If the value of C exceeds a predefined integer count, the process 200 proceeds to step 216. In one embodiment, the predefined integer count may be 2. In other embodiments, the predefined integer count may be a larger integer, in order to achieve a desired amount of statistical confidence when analyzing the data set 22C in the steps outlined below. If, however, the predefined integer is not achieved at step 214, process 200 repeats at step 204.

At step 216, the processor 12 receives instructions for applying an N-of-1 evaluation on the response data 22. The algorithm 20 may be configured to determine the particular N-of-1 technique to apply to the response data 22, based on a family of N-of-1 evaluation techniques that may be recorded in the memory 14. The N-of-1 techniques may be selected based on an optimal method, such as but not limited to PND, PEM, Kendall Tau, and the like, to evaluate segment change on one or more variables at the level of the individual unit patient N. The process 200 proceeds to step 218.

At step 218, the N-of-1 evaluation technique is applied to the response data 22, e.g., the pre-processed time-series response data 22C to determine one or more confidence scores 22D (e.g., IAQ score) associated with the time-series response data 22C. In determining the IAQ scores 22D, the evaluation technique may also take into account one or more items of information stored in the patient profile 22A. The IAQ score 22D is recorded in the database 18 at step 220. The process 200 is configured to repeat at step 204 to receive additional raw response data 22B associated with a new treatment segment. The process 200 may be configured to transmit the IAQ score 22D to any user device 70, 71, wearable device 73, and the like, on-demand. At the completion of step 218, the process 200 also proceeds to step 222.

At step 222, the algorithm 20 may be configured to analyze the response data 22, including the time-series response data 22C, the patient profile response data 22A, the IAQ scores 22D, and the like, in order to identify and assign the individual unit to a segment pertaining to the patient's N treatment response to one or more micro-treatments. The information pertaining to the assigned segments of the individual units may be recorded in the database 18 at step 224. The process 200 may next proceed to step 226.

At step 226, the algorithm 20 may be configured such that a signal S is selectively transmitted to one of the user devices 70, 71 and/or the wearable device 75, via the network 76, in order to generate a graphical user interface (GUI) on a visual display that represents the change of the individual unit and segment, over time. In one non-limiting example, with reference to the Figures, the display may represent the segments along two or more variables, over time, on a GUI or a display screen, and superimpose a visualization of the individual unit's time series data on the time series paths of the segments. As represented in FIGS. 5-40, the visual displays may be configured to essentially create motion pictures representing changes (sequence) of the data, over time. The virtual displays may be generated based on crowd sourcing of the aggregated and replicated N-of-1 experiments (discrete micro-treatments) the application of rules for degree of replication of findings within a particularly similar set of test context that would place the individual unit into the most probably segment.

At step 226, the algorithm 20 may be configured to generate the visual display based on specific data display parameters, received by the processor 12, 68 via a GUI wizard at input 300, to be represented on the visual display. The system 100 provides the GUI wizard to collect, from the user, the requested display and/or animation display parameters in order to determine which data needs to be retrieved from the database and processed to display the requested animation display, with the requested parameters. The unique animation of time series data may include, but should not be limited to, the time-series display of treatment responses for a patient, the time-series display of the IAQ scores 22D, the time-series display of information regarding highly replicated findings as treatment suggestions, an animation display of the time-series progression of the data, and the like. A “wizard” is one or more interactive display screens that present selectable or configurable options to collect information from the user (i.e., patient, caregiver, doctor, and the like) and then use that information to perform some task. Information may be may be collected by the GUI wizard. The information collected may include, but should not be limited a selection of a range of segments to display, a selection of micro-treatment segments to display, a selection level of IAQ to display, a selection of advice on a best next micro-treatment, a selection of data animation attribute groupings, a selection of data animation summaries (i.e., ranges of the subgroups/groups over time), a selection of patient profile attributes, and the like. The method next proceeds to step 228.

At step 228, the algorithm 20 may apply an analysis to determine whether one or more recommended micro-treatments may be available within the data store 18 that would be suitable for trial by the patient N. The determination may be based on what N-of-1 experiments exist within the database 18, by way of recommendations (i.e., machine learning, artificial intelligence, or other algorithms, and the like). An increase in the number of replications aggregate the power of this step in the analysis. Any recommended micro-treatments 22E may be recorded in the database 18 at step 230 for selective retrieval.

Therefore, the treatment system 100 may be configured to provide data processing and evaluation steps that include, but should not be limited to, data acquisition and organization; N-of-1 evaluation system building blocks; N-of-1 aggregation visualization; N-of-1 aggregation segmentation operationalization; and tracking and crowdsourcing N-of-1 aggregation (visualization and animation).

With respect to the data acquisition and organization, the system 100 is configured to accept and utilize all forms of time ordered data (i.e., time series, repeated measures, etc.), independent of the data collection methodology and technology. In one non-limiting example, the system may be configured to accept time series data with varying time collection intervals using either parametric or non-parametric data and will order said data in a pre-defined manner (e.g., standardize, normalize, correct for missing data, local time synchronization, universal time synchronization, etc.).

The N-of-1 evaluation is a system building block. When performing the N-of-1 evaluation, a family of evaluative methods for N-of-1 analysis are applied to the patient N data, based on optimized decision rules for such an application to evaluate segment changes (change on one or more independent variables within the individual unit patient N) under two or more segment conditions. More specifically, a method for performing the N-of-1 analysis is selected to evaluate the effectiveness/ineffectiveness of the discrete micro-treatments, based on the measures (data) recorded at spaced time intervals during the fixed time period of the segment. In one non-limiting example, the fixed time periods are one-month intervals, and the measures per segment are daily. It should be appreciated that intervals having longer or shorter lengths of time and more or less measures per segment may also be used without departing from the scope. The N-of-1 evaluation provides the IAQ score.

N-of-1 aggregation provides a visualization and evaluation of the IAQ score relative to a change in a time series data trend 50 (FIG. 3B for example) of the N-of-1 level data and results, which may be aggregated for two or more individual units patient N. Further, the patient data and/or IAQ scores may evaluated in terms of the degree of co-relationships (e.g. trends) for two or more variables that may also be based on an aggregation of N-of-1 findings.

The N-of-1 aggregation segmentation operationalization is based on optimized decision rules. As such, the system 100 is configured to evaluate and aggregate the results of the N-of-1 evaluation (based on aggregated N-of-1 results) into groups (i.e., “segments”) based at least in part, on unique data attributes of the individual unit patient N (static and/or cross-sectional data), the unique trend over time, and the unique responses to the same or similar segment changes. Further, decision rules may be provided for the aggregation segmentation operationalization of the data and/or IAQ score to optimize the homogeneity within the group and/or heterogeneity between the groups.

The tracking and crowdsourcing or friendsourcing N-of-1 aggregation (i.e., visualization and animation) uses the time series data, renders an animated visualization of the time-series data over time (data in motion) on the display of the GUI. Friendsourcing is similar to crowdsourcing, but use is generally limited to a set of “friends”, or a grouping of selected other patients N. This visualization can be rendered at the entire sample (population) level, segment (group) level, or individual level separately or collectively. Providing such a visualization and underlying evaluation on a display as a GUI will test one or more variables at the level of the individual unit patient N and relative to a defined comparator (e.g., goal, guideline, ideal, population norms, normal limits, etc.) and evaluation of an individual unit patient N trend (and/or outcome), relative to the comparator. As such, a statistical and visual comparison between the individual unit patient N trend over time and the comparator change over time both within and between segments may be realized.

The tracking and crowdsourcing N-of-1 aggregation (i.e., segmentation experimentation) is based on optimized decision rule. As such, the system promotes (i.e., recommends, offers, reinforces) segment changes based on aggregating (dynamic data) to individual units N to further test and validate patterns in segment change.

Crowdsourcing and/or friendsourcing sharing of N-of-1 micro-treatments and IAQ's across the patient N and healthcare provider D community is enabled by the communication interface 72 and the suggestions module 36 to provide opportunity to visualize and identify potentially new micro-treatments that might have high positive outcomes with good statistical confidence from other patient N. The communication interface 72 provides the healthcare providers D and the patients N with the opportunity to add particular micro-treatment to the suggestions module 36, in the event the outcome of a particular micro-treatment was positive. To add the particular micro-treatment to the suggestions module 36, the healthcare provider D and/or the patient P may make a selection on a menu generated by the GUI wizard on the display screen. Alternatively, the system 100 may be configured such that micro-treatments are automatically added to the suggestions module 36 if the micro-treatment results in a certain confidence score. Conversely, the communication interface 72 and suggestions module 36 may also provide the opportunity to identify micro-treatments where the outcome was not positive.

Referring again to FIG. 2, the treatment process 200 executed through the treatment system 100 is configured to evaluate a plurality of time series data and/or repeated measures data (i.e., data that is continuously collected and evaluated over a specified time period), at the individual unit N (i.e., single patient or N-of-1) level of analysis. The treatment process 200 is then configured to detect a change in an individual unit N (i.e., the single patient or N-of-1) under two or more distinct conditions (i.e., a treatment and/or an intervention response by the single patient). The system 100 is configured to apply and evaluate a plurality of N-of-1 “segmented” evaluation method, including, but not limited to, PEM, PND, Kendal Tau; comparing change between two (or more) distinctly characterized segments (e.g., treatment conditions) with at least 2 measures per segment; and animate and visualize the time series “segment change” data over time (e.g., clinical response), via a display on the GUI. It should be appreciated that there may be any number of segments, and the distinctly characterized segments are not limited to being sequentially ordered. As such, the distinctly characterized segments may be spaced, with other distinctly characterized segments in between that are not being evaluated.

The treatment process 200 is also configured to aggregate collective time series “segment change” data, over time, and use a plurality of segmentation identification and evaluation methods to identify unique groupings of individual units N based on decision rules designed to optimize the homogeneity within the group and heterogeneity between the groups both in terms of static (unchanging) attributes and their N-of-1 evaluated change of time. The segmentation identification and evaluation methods may include, but should not be limited to, LGMM, Cluster Analysis, etc.

The treatment process 200 may be configured to evaluate an individual unit patient N relative to the attributes that make up a given segment and place using a plurality of evaluative methods (e.g. nearest neighbor, etc.) to define a membership of the individual unit patient N relative to the defined segments. A time series course of both an individual and their relationship to the unique segments, over time, may be superimposed within animated data displayed on the display.

In another aspect of the disclosure, the treatment process may be configured to evaluate a plurality of cross-sectional data and time-series/repeated measures data (i.e., data continuously collected and evaluated over a specified time period) at the individual unit patient N (single patient) and aggregated (segment) grouping of patient N's level that identifies and evaluates the individual units patient N unique attribute, relative the unique attributes of defined segments (including an overall course). The treatment process 200 is configured to inform the patient (individual unit N) of those self-attributes and the strength of those attributes that contribute to the patient's placement within a specifically defined segment and the contribution of those attributes to a predicted time-series course, based on the segments established course. The N-of-1 change is evaluated within the individual unit N in those attributes contributing to the placement in a particular segment, relative to segment membership, and changed predicted time-series course.

A collective N-of-1 change within a given sample/population is evaluated, based on a defined set of rules, and based on feedback via data, tables, and visualization information regarding highly replicated findings as treatment suggestions for those individual units (patient Ns) from within the larger database that have not yet been exposed to the favorably identified treatment condition(s).

In another aspect of the disclosure, a treatment process 200 is provided for evaluating a plurality of cross-sectional and time-series/repeated measures data (i.e., data that is continuously collected and evaluated over a specified time period) at the individual unit patient N and within small (practice level) patient N groups undergoing similar or competitive treatment options. The treatment process 200 is configured to provide practitioners with standard, but customizable, N-of-1 segment and micro-treatment designs (e.g., ABAB, multiple baseline, etc.) for optimized application of N-of-1 segments, data collection, and evaluation based on, and specific to, a given clinical context for conducting alternative treatment evaluation within a small set of individual units patient Ns. The treatment process 200 may also be configured to provide practice level (or clinician level) evaluation and visualization of treatment responses in each individual unit N (single patient), including the display on a display screen of unique animation of time series data for optimized care.

In some implementations, the computer executable code may include multiple portions or modules, with each portion designed to perform a specific function described in connection with FIGS. 1-4 above. In some implementations, the techniques may be implemented using hardware such as a microprocessor, a microcontroller, an embedded microcontroller with internal memory, application specific integrated-circuit (ASIC), internet of things (IoT) device, or an erasable programmable read only memory (EPROM) encoding computer executable instructions for performing the techniques described in connection with FIGS. 1-4. In other implementations, the techniques may be implemented using a combination of software and hardware.

It should be appreciated that the treatment system 100 and treatment process 200 is not limited to the examples as described herein. Other applications of the system 100 and process 200 are also contemplated, including, but not limited to, use with artificial intelligence (AI) engines to personalize or recommend actions; use with applications to share historical treatment (independent variable) insights on health and life outcomes (dependent variables); use IAQ scores 22D as digital phenotypes to connect with physical phenotypes (e.g., blue eyes, red hair, etc.) and genotype and disease/health history for new level of improved health and life management; use with quadrant or matrices for other multi-dimensional mapping to be displayed on the display screen to see endpoint or data movie (i.e., animation) patterns, and the like; use with multi-variable analysis to see combinations of co-independent variable and/or co-dependent variable relationships; use to add lag and/or lead time analytics; use additional N-of-1 mathematics of known science to offer and graphically display predictive, next-segment or other future segment insights, based on receiving, by the processor 12, 68 via the GUI wizard, data display parameters; use the response data 22, including the IAQ scores 22D and the data movies in conjunction with a digital or personal health/life coach to support behavior change management of the patient N; use with reminders to improve the patient's N treatment (independent variable) plan compliance, and the like.

Time-series data comes in for key health and life attributes/variables. The time-series data may be collected via sensors or digital health diaries on user devices 70, 71 and/or devices 74. As explained above, this time-series response data 22 for the patient N is stored in the database 18 and converted to a time ordered structure (standardize frequency), with a relationship to the segmented interventions (micro-treatments). Then, by way of a non-limiting example, with reference to FIGS. 3A and 3C, two or more of the patient N attributes/variables are plotted against other, as a GUI on the display screen 74, based on the time ordered relationship and unique colors/shading/coding to show transitions of the segmented interventions (micro-treatments). This plotting may be combined with multiple other patients N to see a trajectory of the group and/or sub-groups versus the individual patient N. The data can be displayed as a movie or a snapshot in time (of the data movie), as illustrated in FIGS. 5-40. To improve viewability, the IAQ values 22D may be shown in any color, shade, symbol, code, and the like.

While the best modes for carrying out the disclosure have been described in detail, those familiar with the art to which this disclosure relates will recognize various alternative designs and embodiments that fall within the scope of the appended claims.

1. A method of using a patient treatment system to treat a patient, the method comprising:
receiving, by a computing device, first and second order response data corresponding to a respective first and second micro-treatment prescribed to a patient, wherein the first and second order response data represents results of the respective first and second micro-treatment for the patient at each of a plurality of intervals in time;
wherein the second micro-treatment occurs after the first micro-treatment;
recording the first and second order response data into a database that includes time series response data for each of the first and second micro-treatments;
calculating, by the computing device:
a first data score and a second data score by applying an N-of-1 statistical analysis respectively to each of the first and second order response data, wherein the first and second data scores statistically represent an effectiveness of the respective first and second micro-treatment;
a trend of the first and second data scores; and
a statistical confidence associated with each of the first and second data scores;
recording the first and second data scores into the database;
generating, by the computing device, a graphical user interface on a display screen of a user device, wherein the graphical user interface comprises at least one of:
an effectiveness display that displays at least one of the response level to each of the first and second micro-treatments and a trend line representing the trend of the first and second data scores;
the first and second data scores and a confidence display that displays the statistical confidence associated with each of the first and second data scores; and
first and second graphical elements, wherein the first and second graphical element represent the statistical confidence associated with each of the first and second data scores; and
generating, by the computing device, a graphical user interface on the display screen of the user device comprising at least one third micro-treatment option to be prescribed to the patient.

2. The method of claim 1, wherein the user device is a healthcare provider user device.

3. The method of claim 1, wherein the first and second order response data received by the computing device is received from at least one of a patient user device and a wearable device; and
wherein at least a portion of the first and/or second order response data is collected automatically by the at least one of a patient user device and a wearable device at each of the plurality of intervals in time.

4. The method of claim 1, wherein the first and second micro-treatments each include at least two treatment actions.

5. The method of claim 4, wherein at least one of the at least two treatment actions of the second micro-treatment is different from at least one of the at least two treatment actions of the first micro-treatment.

6. The method of claim 1, wherein generating a graphical user interface further comprises a response display that displays an X-Y plot representing the first order and second order response data at each of the plurality of intervals during the respective first and second micro-treatment.

7. The method of claim 1, further comprising:
receiving, by a computing device, third order response data corresponding a third micro-treatment prescribed to the patient, wherein the third order response data corresponds to the results of the third micro-treatment for the patient at each of a plurality of intervals in time;
recording the third order response data into the database that includes time series response data for the third micro-treatment;
calculating, by the computing device, a third data score, based on an N-of-1 statistical analysis of the third order response data and at least one of the first and second order response data, wherein the third data score statistically represents an effectiveness of the third micro-treatment;
recording the third data score into the database;
wherein the graphical user interface generated on the display screen of a user device further comprises:
a third order response display that displays an X-Y plot representing the third order response data at each of the plurality of intervals during the third micro-treatment;
a confidence that displays a statistical confidence associated with the third order response data; and
at least one fourth micro-treatment option to be prescribed to the patient, based, at least in-part, on the first, second, and third data score of at least one of the first, second, and third order response data.

8. The method of claim 1, further comprising:
recording at least one health attribute of the patient into the database, such that the at least one health attribute is associated with a patient profile of the patient;
recording at least one health condition of the patient into the database, such that the at least one health condition is associated with the patient profile of the patient;
wherein the recording the first and second order response data into a database is further defined as recording the first and second order response data into a database that includes time series response data for each of the first and second micro-treatments, such that the first and second order response data is associated with the patient profile of the patient;
wherein recording the first and second data scores into the database is further defined as recording the first and second order data scores into the database, such that the first and second data scores are associated with the patient profile of the patient.

9. The method of claim 8, wherein the database includes another patient profile corresponding to one other patient, wherein the patient profile of the other patient includes:
a health attribute of the other patient;
a health condition of the other patient;
first and second order response data corresponding to a first and second micro-treatment prescribed to the other patient, wherein the first and second order response data corresponds to the results of the respective first and second micro-treatments at each of a plurality of time intervals; and
first and second data scores that statistically represent an effectiveness of each of the first and second micro-treatments;

10. The method of claim 9, further comprising:
determining, by the computing device, at least one other patient, with a patient profile recorded in the database, having at least one of:
a health attribute equal to the at least one health attribute of the patient,
a health condition equal to the at least one health condition, and
a type of the first and second micro-treatments prescribed to the other patient being the same type of first and second micro-treatments prescribed to the patient;
retrieving, by the computing device from the database, at least one of the first and second order response data corresponding to the results of the respective first and second micro-treatment for the other patient; and
wherein the display of the graphical user interface generated on the display screen of a user device is further defined as a response display that displays an X-Y plot of the patient representing the first order and second order response data at each of the plurality of intervals during the respective first and second micro-treatment and an X-Y plot for the other patient representing the first order and second order response data at each of the plurality of intervals during the respective first and second micro-treatment for the other patient;
wherein the X-Y plot for the patient is graphically distinguished to be different from the X-Y plot for the other patient.

11. The method of claim 10, wherein a response display that displays an X-Y plot of for the patient representing the first order and second order response data at each of the plurality of intervals during the respective first and second micro-treatment and an X-Y plot for the other patient representing the first order and second order response data at each of the plurality of intervals during the respective first and second micro-treatment further includes displaying each data point of the first order and second order data in sequential time series order for the X-Y plot for the patient and for the other patient, simultaneously, such that the display of the X-Y plot for the patient and for the other patient is animated.

12. The method of claim 1, wherein the graphical user interface generated on the display screen of a user device further comprises a change display that displays an X-Y plot of the first data score and the second data score to graphically represent an amount of change of the micro-treatment effectiveness from the first micro-treatment to the second-micro-treatment.

13. The method of claim 12, further comprising calculating, by the computing device, a first delta value representing a difference between the second data score and the first data score, wherein the first delta value represents an effectiveness of the second micro-treatment, as compared with the first micro-treatment; and wherein the graphical user interface generated on the display screen of a user device further comprises a delta display that displays the first delta value.

14. A method of treating a patient with a patient treatment system, the method comprising:
receiving, by a computing device, first and X^{th} order response data corresponding a respective first and X^{th} micro-treatment prescribed to a patient, wherein the first and X^{th} order response data corresponds to the results of the respective first and X^{th} micro-treatment for the patient at each of a plurality of intervals in time;
wherein the X^{th} micro-treatment occurs after the first micro-treatment;
recording the first and X^{th} order response data into a database that includes time series response data for each of the first and X^{th} micro-treatments;
calculating, by the computing device, a first data score and an X^{th} data score by applying an N-of-1 statistical analysis respectively to each of the first and X^{th} order response data, wherein the first and X^{th} data scores statistically represent an effectiveness of the respective first and X^{th} micro-treatments;
calculating, by the computing device, a first-to-X^{th} delta representing a difference between the X^{th} data score and the first data score, wherein the first-to-X^{th} delta represents an amount of change of the micro-treatment effectiveness from the first to the X^{th} micro-treatment; and
generating, by the computing device, a graphical user interface on a display screen of a user device, wherein the graphical user interface comprises a change display that displays an X-Y plot of the first data score and the X^{th} data score to graphically represent an amount of change of the micro-treatment effectiveness from the first micro-treatment to the X^{th} micro-treatment.

15. The method of claim 14, further comprising:
receiving, by a computing device, X^{th-1} order response data corresponding an X^{th-1} micro-treatment prescribed to the patient, wherein the X^{th-1} order response data corresponds to the results of the X^{th-1} micro-treatment for the patient at each of a plurality of intervals in time;
recording the X^{th-1} order response data into the database that includes time series response data for the third micro-treatment;
calculating, by the computing device, a X^{th-1} data score, based on an N-of-1 statistical analysis of the third order response data, wherein the X^{th-1} data score statistically represents an effectiveness of the X^{th-1} micro-treatment;
recording the X^{th-1} data score into the database;
calculating, by the computing device, an X^{th-1}-to-X^{th} delta representing a difference between the X^{th} data score and the X^{th-1} data score, wherein the X^{th-1}-to-X^{th} delta represents an amount of change of the micro-treatment effectiveness from the X^{th-1} micro-treatment to the X^{th} micro-treatment;
wherein the graphical user interface generated on the display screen of a user device further comprises a change display that displays an X-Y plot of at least two of the first data score, the X^{th} data score, and the X^{th-1} data score to graphically represent an amount of change of the micro-treatment effectiveness from the first micro-treatment and the X^{th} micro-treatment and the X^{th-1} micro-treatment and the X^{th} micro-treatment.

16. The method of claim 15, wherein a change display that displays an X-Y plot is further defined as a change display that displays X-Y plots of the first data score and the X^{th} data score and of the X^{th-1} data score and the X^{th} data score to graphically represent an amount of change of the micro-treatment effectiveness from the first micro-treatment and the X^{th} micro-treatment and the X^{th-1} micro-treatment and the X^{th} micro-treatment.

17. The method of claim 14, wherein the first and X^{th} order response data received by the computing device is received from at least one of a patient user device and a wearable device; and
wherein at least a portion of the first and second order response data is collected automatically by the at least one of a patient user device and a wearable device at each of the plurality of intervals in time.

18. A method of treating a patient with a patient treatment system, the method comprising:
recording at least one health attribute and at least one health condition of a patient into a database, such that the at least one health attribute and the at least one health condition is associated with a patient profile of the patient;
recording first and second order response data into a database that includes time series response data for each of a first and second micro-treatment, such that the first and second order response data is associated with the patient profile of the patient;
calculating, by the computing device, a first data score and a second data score by respectively applying an N-of-1 statistical analysis to each of the first and second order response data, wherein the first and second data scores statistically represent an effectiveness of the respective first and second micro-treatment;
recording the first and second data scores into the database, such that the first and second data scores are associated with the patient profile of the patient;
calculating, by the computing device, a first-to-second delta representing a difference between the second data score and the first data score, wherein the first-to-second delta represents an amount of change of the micro-treatment effectiveness from the first to the second micro-treatment;
recording the first-to-second delta into the database, such that the first-to-second delta is associated with the patient profile of the patient;
wherein the database further includes another patient profile corresponding to one other patient, wherein the patient profile of the one other patient includes a health attribute, a health condition, first and second order response data corresponding to a first and second micro-treatment prescribed to the other patient, wherein the first and second order response data corresponds to the results of the respective first and second micro-treatments at each of a plurality of time intervals, and first and second data scores that statistically represent an effectiveness of each of the first and second micro-treatments for the other patient;
generating, by the computing device, a graphical user interface on a display screen of a user device, wherein the graphical user interface comprises a change display that displays an X-Y plot of for the patient representing the first order and second order response data at each of the plurality of intervals during the respective first and second micro-treatment and that displays an X-Y plot for the other patient representing the first order and second order response data at each of the plurality of intervals during the respective first and second micro-treatment.

19. The method of claim 18, wherein a change display is further defined as displaying each data point of the first order and second order data for each of the patient and the other patient is simultaneous, and in sequential time series order, such that the display of the X-Y plot for the patient and for the other patient is animated to visually compare the patient response to the micro-treatments to the other patient response to the micro-treatment during the respective time series.

20. The method of claim 18, wherein the database is further defined as including other patient profiles of a plurality of other patients;
wherein the display of the graphical user interface generated on the display screen of a user device further includes a graphical user interface (GUI) wizard presenting a menu of selectable items to selectively search for other patients in the database at least one selectable, wherein the selectable items include at least one of a value associated with a health attribute, a health condition, a value associated with a data score, a value associated with a delta between two micro-treatments; and
wherein the method further includes searching the database, by the computing device, to find another patient profile containing data matching at least on selectable item selected by a user.
